(19) United States
(12) Patent Application Publication
(10) Pub. No.: US 2021/0010030 A1
(43) Pub. Date: Jan. 14, 2021

(54) METHOD FOR REPROGRAMMING SOMATIC CELLS

(71) Applicants: NSERM (INSTITUT NATIONAL DE LA SANTÉ ET DE LA RECHERCHE MÉDICALE), Paris (FR); ECOLE NORMALE SUPÉRIEURE DE LYON, Lyon (FR); CENTRE NATIONAL DE LA RECHERCHE SCIENTIFIQUE - CNRS, Paris (FR); INSTITUT NATIONAL DE LA RECHERCHE POUR L'AGRICULTURE, L'ALIMENTATION ET L'ENVIRONNEMNT, Paris (FR); UNIVERSITE CLAUDE BERNARD LYON 1, Villeurbanne (FR)

(72) Inventors: Bertrand PAIN, Bron (FR); Noémie AURINE, Lyon (FR); Camille BAQUERRE, Bron (FR); Branka HORVAT, Lyon (FR)

(21) Appl. No.: 17/040,246

(22) PCT Filed: Mar. 22, 2019
(86) PCT No.: PCT/EP2019/057324

§ 371 Date: Sep. 22, 2020

(30) Foreign Application Priority Data
Mar. 22, 2018 (EP) 18305321.4

(51) Int. Cl. C12N 15/86; C12N 5/074; A61K 35/33

(52) U.S. Cl. CPC C12N 15/86; C12N 2506/1307; A61K 35/33; C12N 5/0696

(57) ABSTRACT

The disclosure relates to a method for reprogramming somatic cells from mammalian cells including human, bat and equine cells. The inventors have identified an original combination of reprogramming factors for use in methods for reprogramming somatic cells into stem cells from various species, such as bat, bovine, equine and human species. In particular, the disclosure relates to an in vitro method for preparing stem cells reprogrammed from mammalian somatic cells, said method comprising culturing mammalian somatic cells and expressing at least the following combination of reprogramming factors: (i) a reprogramming factor encoded by ESRRB gene, (ii) a reprogramming factor encoded by CDX-2 gene, and, (iii) a reprogramming factor encoded by c-MYC gene, under appropriate conditions for reprogramming said mammalian somatic cells into stem cells.

The disclosure relates to a method for reprogramming somatic cells from mammalian cells including human, bovine, bat and equine cells.

In particular, the disclosure relates to an in vitro method for preparing stem cells from mammalian somatic cells, said method comprising culturing mammalian somatic cells and expressing at least the following combination of reprogramming factors:
(i) a reprogramming factor encoded by ESRRB gene,
(ii) a reprogramming factor encoded by CDX-2 gene, and,
(iii) a reprogramming factor encoded by c-MYC gene,
   under appropriate conditions for reprogramming said mammalian somatic cells into stem cells.

### BACKGROUND

Somatic reprogramming refers to the process consisting of adding a combination of genes—generally transcription factors—that induce the change from a somatic cell to a stem cell, that has then been qualified as an ‘iPS’ cell for induced pluripotent stem cell (Takahashi and Yamanaka, 2006, 2016; Karagiannis and Eto, 2016). For example, the combination of four transcription factors, Oct4, Sox2, Klf4 and c-Myc—OSKM or Yamanaka combination—enables a fibroblast to become an iPS cell in many species. Other different gene combinations have also been identified such as the OSNL cocktail (Oct4, Sox2, Nanog and Lin28), called the Thomson combination (Yu et al., 2007) in the presence or absence of other transcription factors that directly participate in or increase the efficiency of the reprogramming process (Hochedlinger & Plath, 2009, Stadtfeld & Hochedlinger, 2010), such as the Nr5a2 (Heng et al., 2009), ESRRB (Feng et al., 2009, Yang et al., 2017), ZIC3 (Declercq et al., 2013), TBX3 (Han et al., 2010), miR302, (Anokye-Danso et al., 2011) and Zpf296 (Fichedick et al., 2012) genes.

More recently, the JARID2, PRDM14, ESRRB and SALL4A genes have been demonstrated to participate in increasing the efficiency of somatic reprogramming (Iseki et al., 2016). But in all cases, there was no major substitution in the original OSKM combination.

The consensual definition of a reprogrammed cell is obtained from a somatic cell or a less differentiated precursor of a cell with the characteristics of an ES and/or ES-like cell: self-renewal and differentiation in the different embryonic layers. The presence and the expression of transgenes that induce reprogramming must be transient and the endogenous pluripotent genes alone must essentially maintain self-renewal and differentiation properties of iPS cells. There is a great deal of interpretation about the latter property because it is not usually observed in most published examples in species other than rodents, man and non-human primates.

The reprogramming approach was first observed in the mouse model, and was then successfully used on human fibroblasts, on the rat, on cells of non-human primates and with more or less success in a large number of mammalian species including the rabbit (Honda et al, 2013; Osteil et al, 2013), sheep, bovines, (Liu et al, 2012; Sumer et al, 2011), pigs (Ezashi et al, 2009), horses (Nagy et al., 2011), dogs (Shimada et al, 2010) and even in some endangered species (Verma et al, 2013). It is sometimes difficult to check in publications if endogenous genes no longer express themselves and therefore if the cells have been well reprogrammed. In particular, the genetic stability of the presented cells is not illustrated and the question of their establishment in the long term remains open in the absence of a growth curve.

Only one publication—Mo et al., 2014—mentions the preparation of iPS cells in insectivorous bats Myotis lucifugus with the combination of Oct4, Sox2, Klf4, Nanog, cMyc, Lin28, Nr5a2, and miR302/367 genes.

The inventors have now identified an original combination of reprogramming factors for use in methods for reprogramming somatic cells into stem cells from various species, including bat, bovine, equine and human species.

### SUMMARY

The present disclosure relates to an in vitro method for preparing stem cells from mammalian somatic cells, said method comprising culturing mammalian somatic cells and expressing at least the following combination of reprogramming factors:
a reprogramming factor encoded by ESRRB gene,
a reprogramming factor encoded by CDX-2 gene, and,
a reprogramming factor encoded by c-MYC gene,
   under appropriate conditions for reprogramming said mammalian somatic cells into stem cells. In specific embodiment, the method does not include a step of exogenous expression of at least one of the following reprogramming factors: Oct4, Klf4, Sox2, Lin28 and Nanog. Typically, said mammalian somatic cells may be selected from the group consisting of bovine, equine, human and bat somatic cells.

In other specific embodiments, that may be combined with the previous embodiments, said mammalian somatic cells are selected from primary cells of blood, bone marrow, adipose tissue, skin, hair, skin appendages, internal organs such as heart, gut, lung, trachea, kidney or liver, mesenchymal and parenchymal tissues containing primary fibroblasts, muscle, bone, cartilage or skeletal tissues.

In other specific embodiments, that may be combined with the previous embodiments, said conditions for reprogramming include the exogenous expression of the reprogramming factors particularly between at least 5 to 10 days for the bovine, at least 10 to 20 days for the human and horse and between at least 30 to 50 days for the bat somatic cells.

In other specific embodiments, that may be combined with the previous embodiments, said conditions for expressing the reprogramming factors comprise
introducing one or more expression vectors comprising the coding sequences of said combination of reprogramming factors into said somatic cells; or,
directly delivering an effective amount of each reprogramming factor of the combination or their precursor RNA into said somatic cells.

For example, the method may include a step of transfecting said somatic cells with an episomal, viral or transposon vector, or a combination of episomal, viral or transposon vectors, comprising the coding sequence of each of the reprogramming factors respectively: ESRRB, CDX-2 and c-MYC.

The disclosure also pertains to the stem cells obtainable according to the method as described above. In specific embodiments, said stem cells are either
bovine stem cells and are characterized by the expression of the telomerase gene TERT and at least one or more of the following genes: CDL1, DAB1, GMPR, FOLR1, PIWIL1, TOPAZ;
bat stem cells and are characterized by the expression of the telomerase gene TERT, and EMA1, and at least one or more of the following genes: NELL2, SIDT1, NWD2, SOX2, TBR1;
human stem cells and are characterized by the expression of the telomerase gene TERT and at least one or more of the following genes: KLF5, GATA4.

Advantageously, said stem cells are characterized in that their susceptibility to virus infection is increased up to at least 50%, particularly at least 90%, as compared to parental somatic cells from which they have been obtained, as measured in vitro by an infection test.

In specific embodiments, the disclosure relates to the stem cells identified as “NA14-R3_PTC+EMC”, “H81-6_BEF+EMC”, “Clone35 HEF+EMC”, as deposited at CNCM on Mar. 14, 2018, under deposit number CNCM 1-5295, 1-5296 and 1-5297 respectively, in the name of
(i) Institut National de la Santé et de la Recherché Medicale, 101 rue de Tolbiac, 75013 Paris,
(ii) Institut National de la Recherche Agronomique, 147 rue de l'Université, 75338 Paris Cedex 07,
(iii) Université Claude Bernard Lyon, 43 Boulevard du 11 Nov. 1918, 69100 Villeurbanne.

The disclosure also relates to a kit for reprogramming somatic cells, said kit comprising:
(i) one or more expression vectors for the exogenous expression of ESRRB, CDX-2 and c-MYC genes in mammalian cells, and
(ii) optionally, buffers, growth factors, antibiotics and other chemicals and/or culturing media.

In specific embodiments of the kit, said expression vectors are selected from viral vectors, episomal vectors and transposon vectors.

The disclosure further relates to the use of the stem cells as described above, for cell therapy, for regenerative therapy, for screening and testing drugs, for replicating and testing the virulence of pathogens, in particular, viral pathogens, or as a research tool for studying infection and propagation of viral pathogens.

### DETAILED DESCRIPTION

A first aspect of the disclosure relates to an in vitro method for preparing stem cells, namely, reprogrammed stem cells, from mammalian somatic cells, said method comprising culturing mammalian somatic cells and expressing at least the following combination of reprogramming factors:
a reprogramming factor encoded by ESRRB gene,
a reprogramming factor encoded by CDX-2 gene, and,
a reprogramming factor encoded by c-MYC gene,
   under appropriate conditions for reprogramming said mammalian somatic cells into stem cells.

As used herein, the term “stem cell” refers to a cell that has an ability for self-renewal and an endogenous telomerase activity. Particularly, the stem cell has at least the following properties:
(i) an ability for self-renewal,
(ii) a doubling time at least twice faster than the corresponding fibroblast, typically an average doubling time comprised between 18 to 24 hours for human stem cells (as compared to 30 to 50 hours or more for non-senescent fibroblasts prior to reprogramming),
(iii) a typical cellular cycle with a shorter phase G0/G1 as compared to the duration of the same phase as observed in fibroblasts, and
(iv) an endogenous telomerase activity.

The reprogrammed stem cells refers to the stem cells as obtainable or obtained by the above described method comprising culturing mammalian somatic cells and expressing at least the following combination of reprogramming factors:
a reprogramming factor encoded by ESRRB gene,
a reprogramming factor encoded by CDX-2 gene, and,
a reprogramming factor encoded by c-MYC gene,
   under appropriate conditions for reprogramming said mammalian somatic cells into stem cells.

The stem cell may be pluripotent with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

Alternatively, the stem cell is capable of differentiating only in certain types of tissues.

The reprogrammed stem cells according to the present disclosure are obtained by artificial means (reprogramming method) and are non-naturally occurring stem cells.

Further characterization of the stem cells of the present disclosure will be described below.

The term “reprogramming” refers to the process of changing the fate of a somatic cell into that of a different cell type, caused by the expression of a small set of factors (or reprogramming factors) in the somatic cells. For example, methods for reprogramming fibroblast cells to stem cells by expressing ectopically Oct3/4, Sox2, c-Myc and Klf4 have been described by Takahashi and Yamanaka, 2006.

In specific embodiments, a “reprogramming factor” is a transcription factor, which can be used to reprogram a target cell.

### The Cells for Use in the Method of the Present Disclosure

The cells for use as a starting material in the reprogramming method of the present disclosure are somatic mammalian cells, typically primary somatic mammalian cells.

As used herein, the term “somatic cells” refers to a given cell lineage, other than stem cells, pluripotent stem cells or germinal cells. By definition, the somatic cells do not express at least the following markers: OCT4, SOX2, KLF4, NANOG, ESRRB.

The somatic cells may be obtained from mammal species, and for examples from rodent, ruminant, equine, porcine, bats, lagomorphs, non-human primate or human species, more particularly from human, bovine, bats or equine species. Typically, the somatic cells for use in the method of the disclosure may be obtained from example from human, mice, rats, cows, horses, sheep, pigs, goats, camels, antelopes, dogs, cats and bats.

The somatic cells may be obtained from various tissues. They may also be obtained from living (biopsies) or from frozen tissues of living or dead animals conserved in adapted conditions.

In one specific embodiment, said somatic cells are obtained from primary cells of blood, bone marrow, adipose tissue, skin, hair, skin appendages, internal organs such as heart, gut or liver, mesenchymal tissues, muscle, bone, cartilage or skeletal tissues.

Methods to obtain samples from various tissues and methods to establish primary cells are well-known in the art (see e.g. Jones and Wise, Methods Mol Biol. 1997).

The method usually includes a step of collecting the cells from a biopsy, dissociating the cells with appropriate enzymes and suspending the dissociated cells (primary cells) in an appropriate medium for in vitro culturing.

### The Combination of Reprogramming Factors for Use in the Method of the Present Disclosure

One essential feature of the present method is the use of the following combination of at least the following reprogramming factors:
i. a reprogramming factor encoded by ESRRB gene,
ii. a reprogramming factor encoded by CDX-2 gene,
iii. a reprogramming factor encoded by c-MYC gene.

The combination of reprogramming factors may consist for example of the combination of the 3 reprogramming factors as encoded by human ESRRB, CDX-2 and c-MYC genes. In one preferred embodiment, the method does not include a step of exogenous expression of one of the following reprogramming factors: Oct4, Klf4, Sox2, Lin28 and Nanog. ESRRB (EStrogen Related Receptor Beta) is the gene encoding Estrogen-related receptor beta (ERR-β), also known as NR3B2 (nuclear receptor subfamily 3, group B, member 2), in humans. Exemplary ESRRB gene is the human ESRRB gene (Genbank accession number NM_004452), the bat (P. vampyrus) ESRRB gene (Genbank accession number XM_011357045.1) or the horse (E. caballus) ESRRB gene (Genbank accession number ENSECAT000000011841.1) or variants thereof that encode similar ERR-β transcription factor activity (e.g. within at least 50%, 80%, 90% or 100% activity compared to wild type ERR-β as measured by methods known in the art). In some embodiments, ERR-β variants have at least 90% amino acid sequence identity across their whole sequence compared to the naturally occurring ERR-β polypeptide.

CDX2 is the gene encoding the homeobox protein CDX-2. This gene is a member of the caudal-related homeobox transcription factor family that is expressed in the nuclei of intestinal epithelial cells. Exemplary CDX2 genes are the human CDX2 gene (Genbank accession number NM_001256.3), the bat (P. vampyrus) CDX2 gene (Genbank accession number XM_011360237.1) or the horse (E. caballus) CDX2 gene (Genbank accession number ENSECAT000000000573.1) or variants thereof that encode similar CDX-2 transcription factor activity (e.g. within at least 50%, 80%, 90% or 100% activity compared to wild type CDX-2 as measured by methods known in the art). In some embodiments, CDX-2 variants have at least 90% amino acid sequence identity across their whole sequence compared to the naturally occurring CDX-2 polypeptide.

Exemplary c-Myc proteins are the proteins encoded by the murine c-MYC gene (Genbank accession number NM_010849), the human c-MYC gene (Genbank accession number NM_002467), the bat (P. vampyrus) c-MYC gene (Genbank accession number XM_011360819.1) and the horse c-MYC gene (Genbank accession number ENSECAT00000023507.1) or variants thereof that encode similar c-Myc transcription factor activity (e.g. within at least 50%, 80%, 90% or 100% activity compared to wild type c-MYC as measured by methods known in the art). In some embodiments, c-Myc have at least 90% amino acid sequence identity across their whole sequence compared to the naturally occurring c-Myc polypeptide. N-Myc or L-Myc may also be used as possible reprogramming factor replacing c-Myc.

POU5F1 (POU domain, class 5, transcription factor 1) also known as Oct3/4 is one representative of Oct family. The absence of Oct3/4 in Oct-3/4+ cells, such as blastomeres and embryonic stem cells, leads to spontaneous trophoblast differentiation, and presence of Oct-3/4 thus gives rise to the pluripotency and differentiation potential of embryonic stem cells. Exemplary Oct3/4 proteins are the proteins encoded by the murine Oct3/4 gene (Genbank accession number NM_013633) and the human OCT3/4 gene (Genbank accession number NM_002701)

The terms “Oct3/4”, “Oct4,” “OCT4,” “Oct4 protein,” “OCT4 protein” and the like thus refer to any of the naturally-occurring forms of the Octomer 4 transcription factor, or variants thereof that maintain Oct4 transcription factor activity (e.g. within at least 50%, 80%, 90% or 100% activity compared to wild type OCT4 as measured by methods known in the art). In some embodiments, variants have at least 90% amino acid sequence identity across their whole sequence compared to the naturally occurring OCT4 polypeptide. In other embodiments, the OCT4 protein is the protein as identified by the Genbank reference ADW77327.1.

Exemplary SOX2 proteins are the proteins encoded by the murine Sox2 gene (Genbank accession number NM_011443) and the human SOX2 gene (Genbank accession number NM_003106).

The terms “Sox2,” “SOX2,” “Sox2 protein,” “SOX2 protein” and the like as referred to herein thus includes any of the naturally-occurring forms of the Sox2 transcription factor, or variants thereof that maintain Sox2 transcription factor activity (e.g. within at least 50%, 80%, 90% or 100% activity compared to wild type Sox2 as measured by methods known in the art). In some embodiments, variants have at least 90% amino acid sequence identity across their whole sequence compared to the naturally occurring SOX2 polypeptide. In other embodiments, the SOX2 protein is the protein as identified by the NCBI reference NP_003097.1.

Exemplary KLF4 proteins are the proteins encoded by the murine Klf4 gene (Genbank accession number NM_010637) and the human KLF4 gene (Genbank accession number NM_004235).

The terms “KLF4,” “KLF4 protein” and the like as referred to herein thus includes any of the naturally-occurring forms of the KLF4 transcription factor, or variants thereof that maintain KLF4 transcription factor activity (e.g. within at least 50%, 80%, 90% or 100% activity compared to wild type KLF4 as measured by methods known in the art). In some embodiments, variants have at least 90% amino acid sequence identity across their whole sequence compared to the naturally occurring KLF4 polypeptide. In other embodiments, the KLF4 protein is the protein as identified by the NCBI reference NP_004226.3.

Exemplary NANOG is the protein encoded by murine gene (Genbank accession number XM_132755) and human NANOG gene (Genbank accession number NM_024865).

The term “Nanog” or “nanog” and the like as referred to herein thus includes any of the naturally-ocurring forms of the Nanog transcription factor, or variants thereof that maintain Nanog transcription factor activity (e.g. within at least 50%, 80%, 90% or 100% activity compared to wild type Nanog as measured by methods known in the art). In some embodiments, variants have at least 90% amino acid sequence identity across their whole sequence compared to the naturally occurring NANOG polypeptide. In other embodiments, the NANOG protein is the protein as identified by the NCBI reference NP_079141.

The term “LIN28” or “LIN-28 homolog A” is a protein that is encoded by the LIN28 gene in humans. It is a marker of undifferentiated human embryonic stem cells and encodes a cytoplasmic mRNA-binding protein that binds to and enhances the translation of the IGF-2 (Insulin-like growth factor 2) mRNA. Lin28 has also been shown to bind to the let-7 pre-miRNA and block production of the mature let-7 microRNA in mouse embryonic stem cells. Yu et al. demonstrated that it is a factor in iPSCs generation, although it is not mandatory³. Exemplary LIN28 is the protein encoded by murine gene (Genbank accession number NM_145833) and human LIN28 gene (Genbank accession number NM_024674).

The term “LIN28” or “LIN28 homolog A” and the like as referred to herein thus includes any of the naturally-occurring forms of the Lin28 transcription factor, or variants thereof that maintain Lin28 transcription factor activity (e.g. within at least 50%, 80%, 90% or 100% activity compared to wild type Lin28 as measured by methods known in the art). In some embodiments, variants have at least 90% amino acid sequence identity across their whole sequence compared to the naturally occurring LIN28 polypeptide. In other embodiments, the LIN28 protein is the protein as identified by the NCBI reference NP_078950.

As used herein, the percent identity between the two amino-acid sequences is a function of the number of identical positions shared by the sequences (i. e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino-acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4.

The skilled person may select other corresponding reprogramming factors originating from other mammals, such as mice, rats, cows, horses, sheep, pigs, goats, camels, antelopes, dogs, cats and bats. In some specific embodiments, the skilled person may select the corresponding reprogramming factor from the same species as the target cells used as starting material in the method of the disclosure.

In certain embodiments, the target cells used as starting material are bat cells which are known as virus reservoir, such as Ebola, Marburg virus etc. Typically, this includes bat cells of the genus Pteropus, but also of the genus Roussetus, such as Roussetus aegyptus, known as reservoir virus for Marburg virus, or one of the following species: Hypsignathus monstrosus, Epomops franqueti, Myonycteris torquata, this list being not limitative.

### Conditions for Expressing the Combination of Reprogramming Factors

Any conditions available in the art for expressing a reprogramming factor in the somatic cell can be used in the methods of the disclosure, as long as such conditions result in the presence of reprogramming factor in an appropriate amount and duration for reprogramming said somatic cells to stem cells.

The expression of the reprogramming factors in the cells may be stable or transient. It may also be inducible.

Various methods for transient expression of reprogramming factors have been described in the art. For a review, see Hanna J H, Saha K, Jaenisch R. Cell. 2010 Nov. 12; 143(4):508-25; or, Sheng Ding. Trends in Pharmacological Sciences Volume 31, Issue 1, January 2010, Pages 36-45; and, Feng et al. Cell Stem Cell. 2009 Apr. 3; 4 (4):301-12.

In preferred embodiments, the following alternative conditions may be used for expressing the reprogramming factors:
(i) enhancing endogenous expression of the gene encoding said reprogramming factor,
(ii) allowing exogenous expression of said reprogramming factor by introducing an expression vector comprising a coding sequence of said reprogramming factor operably linked to control sequences into the somatic cell, or
(iii) delivering an appropriate amount of said reprogramming factor or its precursor RNA into the somatic cells.

In another embodiment, one or more expression vectors are used which comprise the coding sequences of the combination of reprogramming factors, for example, ESRRB coding sequence, CDX-2 coding sequence, and, c-MYC coding sequence and/or coding sequences having at least 60%, 70%, 80%, 90% or 95% identity to the corresponding native coding sequences of ESRRB, CDX-2 and c-MYC, while maintaining similar transcription factor activity.

As used herein, the term “coding sequence” relates to a nucleotide sequence that upon transcription gives rise to the encoded product. The transcription of the coding sequence in accordance with the present disclosure can readily be effected in connection with a suitable promoter. Particularly, the coding sequence corresponds to the cDNA sequence of a gene that gives rise upon transcription to a reprogramming factor. In specific embodiments, wherein the cDNA sequence encodes a reprogramming factor originating from a different species as compared to the somatic cell to be reprogrammed, codon optimized sequence may be used.

The percent identity between two nucleotide sequences may be determined using for example algorithms such as the BLASTN program for nucleic acid sequences using as defaults a word length (W) of 11, an expectation (E) of 10, M=5, N=4, and a comparison of both strands.

Expression vectors for exogenous expression of the reprogramming factors may be, for example, plasmid vector, cosmid vector, bacterial artificial chromosome (BAC) vector, transposon-based vector (such as PiggyBac) or viral vector.

In one specific embodiment, the expression vectors used for increasing expression of said reprogramming factors are viral vectors. Examples of such viral vectors includes vectors originated from retroviruses such as HIV (Human Immunodeficiency Virus), MLV (Murine Leukemia Virus), ASLV (Avian Sarcoma/Leukosis Virus), SNV (Spleen Necrosis Virus), RSV (Rous Sarcoma Virus), MMTV (Mouse Mammary Tumor Virus), etc, lentivirus, Adeno-associated viruses, and Herpes Simplex Virus, but are not limited to.

Methods for generating induced pluripotent stem cells based on expression vectors encoding reprogramming factors have been described in the Art, see for example WO2007/69666, EP2096169-A1 or WO2010/042490.

Typically, the coding sequence of any reprogramming factors as used in the method of the disclosure, for example, ESRRB coding sequence, CDX-2 coding sequence, and c-MYC coding sequence, may be operably linked to control sequences, for example a promoter, capable of effecting the expression of the coding sequence in the somatic cell. Such expression vector may further include regulatory elements controlling its expression, such as a promoter, an initiation codon, a stop codon, a polyadenylation signal and an enhancer. The promoter may be constitutive, or inducible. The vector may be self-replicable or may be integrated into the DNA of the host cell.

Alternatively, the vector for exogenous expression is a viral vector and viral particles are produced and used to introduce the coding sequence of said reprogramming factors into said somatic cells. The term « viral particles » is intended to refer to the particles containing viral structural proteins and a sequence coding said reprogramming factors.

Viral particles may be prepared by transforming or transfecting a packaging cell with a viral vector carrying the nucleotide coding sequences of said combination of reprogramming factors.

In a specific embodiment, the expression vectors are transposon-based vectors, typically inducible transposon-based vectors. The somatic cell population may then be transfected using the expression vectors as described above. The term “transfection” or “transfecting” refers to a process of introducing nucleic acid molecules into a cell. The nucleic acid molecules may be gene sequences encoding complete proteins or functional portions thereof. Any appropriate transfection method is useful in the methods described herein.

Incorporating the coding sequence and its control sequences directly into the genome of the somatic cells may cause activating or inactivating mutations of oncogenes or tumor suppressor genes, respectively. For certain applications, in particular medical applications, it may be required to avoid any genetic modifications of the target cells. Accordingly, in a specific embodiment, the reprogramming factors, for example, ESRRB, CDX-2 and c-MYC, or corresponding coding DNA or RNA, are introduced into the somatic cells without integration of exogenous genetic material in the host genomic DNA, i.e. without introduction of the nucleotide sequence in the cell's genome.

An expression vector such as a plasmid or transposon-based vector can be delivered into said cells for ectopic expression of the reprogramming factor, in the form of naked DNA. Alternatively, RNAs coding for said reprogramming factors either chemically modified or not, can be introduced into the cells to reprogram them (see for example Warren L, et al, 2010, Cell Stem Cell. November 5; 7 (5):618-30).

Other expression vectors have been described for example in WO 2009115295.

These nucleic acids can be delivered into the somatic cells with the aid, for example, of a liposome or a cationic polymer, for example, using conventional transfection protocols in mammalian cells.

In particular, appropriate transfection methods that do not use viral DNA or viral particles as a delivery system to introduce the nucleic acid molecules into the somatic cell may be used in the methods described herein. Exemplary transfection methods include without limitation calcium phosphate transfection, liposomal transfection, nucleofection, sonoporation, transfection through heat shock, magnetofection and electroporation. In some embodiments, the nucleic acid molecules are introduced into the target cells using electroporation following standard procedures well known in the art.

Alternatively, the reprogramming factor protein or fragments thereof showing similar properties to the intact proteins with respect to the reprogramming of target cells can be delivered into said target cells with the aid of chemical carriers such as cell-penetrating peptides including, without limitation, penetratin or TAT-derived peptides.

Methods to improve efficiency of the generation of stem cells have also been described in the Art. In particular, in the method of the disclosure, introduction and/or addition of various generation efficiency improving agents may be performed. Examples of substance for improving generation efficiency include, without limitation, histone deacetylase inhibitors (such as for example valproic acid, trichostatin A, sodium lactate, MC1293 and M344), and nucleic acid expression inhibitors such as siRNAs and shRNA for HDAC, and G9a histone methyltransferase inhibitors, and nucleic acid expression system inhibitors such as siRNA and shRNA for G9a (see also Feng et al., 2009).

In one specific embodiment, the methods according to the disclosure do not comprise any step of exogenous expression of one of the following reprogramming factors: OCT4, KLF4, SOX2, LIN28 and NANOG. Typically, none of at least OCT4 or KLF4 are expressed in the stem cells of the present disclosure, contrary to iPS cells obtained by the conventional OSKM combination.

Advantageously, the expression vector includes an inducible system, so that expression is controlled, for example by the addition of a component in the culture medium. Accordingly, in one specific embodiment, the expression of the genes encoding the reprogramming factors is induced by the presence of inducer compound, such as, for example doxycycline. In specific embodiments, the expression of the three genes, ESRRB, CDX-2 and c-MYC is under the control of inducible promoters, particularly the same inducible promoters are used so that the three promoters are induced simultaneously by the same inducer compound.

The somatic cells are cultured under appropriate conditions in a culture medium. The skilled person will be able to select an appropriate culture medium, particularly, liquid medium, for growth of the cells in vitro, under optimized conditions, in particular, regarding pH, temperature, and CO₂ concentration.

Examples of culture media appropriate for mammalian cells include
a fibroblast medium, composed of DMEM/HamF12 medium with 10% fetal bovine serum (FBS) 10,000 U/1,000 U Penicillin/streptomycin stock solution and 20 mM glutamine
a ESM medium composed of DMEM with 10% FBS, 1,000U/1,000U Penicillin/streptomycin stock solution, 20 mM glutamine, 10 mM sodium pyruvate, 0.1 mM β-mercaptoethanol in which 5 ng/mL of basic Fibroblast growth factor (bFGF) and 1 μg/mL. Doxycycline as the promoter of the transgene expression of the inducible vector may be added
an Epi medium composed of a volume/volume mix of DMEM/HamF12 and Neurobasal medium supplemented with 1× of N2 supplements, 1× of B27 supplements, 1,000 U/1,000 U Penicillin/streptomycin stock solution, 0.005% Bovine serum albumin (BSA), 0.1 mM β-mercaptoethanol in which 5 ng/mL of bFGF, 5 ng/mL of Activin A and 1 μg/mL. Doxycycline as the promoter of the transgene expression of the inducible vector may be added

The expression of the genes encoding the reprogramming factors is particularly transient. For example, when using an inducible agent, said agent is particularly removed from the medium so that the expression of the genes ESRRB, CDX-2 and c-MYC decrease progressively.

Expression of the reprogramming factors should be carried out during a time sufficient to enable the reprogramming of the somatic cells to stem cells. Typically, the skilled person may follow the morphological change and reprogramming of the cells by visualising, via a microscope, growth and viability of the cells, and will be able to determine whether expression should be pursued or stopped.

For example, the step of culturing the somatic cells is carried out particularly before the five first cell generations of the primary cells once platted in in vitro culture, and ten generations at most. The step of expressing the genes ESRRB, CDX-2 and c-MYC is carried out particularly during a period of time of at least five to fifty days, for example, at least 5 to 10 days for bovine, at least 10 to 20 days for the human and horse, and at least 30 to 50 days for bats, by adding doxycycline in the culture medium for allowing the expression of the transgenes. The timing of the emergence of the morphological changes depends from one species to another.

### Compositions Comprising Stem Cells Obtainable from the Methods of the Disclosure

The disclosure further relates to a cell-based composition comprising the reprogrammed stems cells as described in the present disclosure, i.e. stem cells obtainable from the method as described above.

Reprogramming somatic cells by expressing CDX-2, c-MYC, and ESRRB can be visualised by the following dramatic changes: transduced cells become smaller and more refracting to the light microscope with a limited cytoplasm, a large nucleus and a higher nucleo-cytoplasmic ratio. Those cells grow then in round colonies and faster than the fibroblasts.

The reprogrammed stem cells can be maintained with the same phenotype for at least 20 passages, particularly between 30 to 45 passages, corresponding to at least 80 generations, typically between 100 to 120 passages in a suitable medium.

As used herein, the term “passage” designate the step of detaching the cells from their support (by means of an enzyme or cocktail of enzymes) and diluting the cells in the culture medium prior to their seeding on a new support for growth. Typically, the cells are numbered after their detachment to get a certain ratio of cells/cm² in petri dish.

Particularly, the stem cells of the present disclosure as induced by ECM combination of reprogramming factors have the following phenotypic features:
expression of telomerase activity,
distribution of cell cycle phases with less than 35% of the cells in G0/G1 phase
no endogenous expression of OCT4, SOX2 and NANOG,
Expression of the following genes: TERT gene,

They may also be characterized by their typical morphology of small round or ovoid cells with an important nucleocytoplasmic ratio,

Remarkably, the bovine reprogrammed stem cells according to the present disclosure are characterized by the expression of one or more of the following genes: CDL1, DAB1, GMPR, FOLR1, PIWIL1, and TOPAZ. Typically, the bovine reprogrammed stem cells express at least two, three, four, five or at least six of these genes.

The bat reprogrammed stem cells according to the present disclosure are characterized by the expression of one or more of the following genes: NELL2, SIDT1, NWD2, SOX2, TBR1. Typically, the bat reprogrammed stem cells express at least two, three, four, five, or at least six of these genes. The bat reprogrammed stem cells according to the present disclosure are also recognized by EMA-1 antibodies (which is known for characterizing murine ES stem cells), however, such bat stem cells are negative for SSEA1 marker (which is the marker for murine ES stem cells).

The human reprogrammed stem cells are characterized by the expression of one or more of the following genes: KLF5, GATA4.

The following table 1 describes the above genes as possibly expressed in the stem cells of the present disclosure.

| Gene Name | Species | Gene ID |
|---|---|---|
| KLF5 | Human | 688 |
| GATA4 | Human | 2626 |
| TERT | Human | 7015 |
| TERT | Bovine | 51884 |
| TERT | Bat | 102895256 |
| TERT | Horse | 100630695 |
| CDL1 | Bovine | 504415 |
| DAB1 | Bovine | 538818 |
| GMPR | Bovine | 533000 |
| FOLR1 | Bovine | 539750 |
| PIWIL1 | Bovine | 537833 |
| TOPAZ | Bovine | 100296400 |
| NELL2 | Bat | 105297489 |
| SIDT1 | Bat | 105288962 |
| NWD2 | Bat | 105288962 |
| SOX2 | Bat | 105307378 |
| TBR1 | Bat | 105291047 |

The stem cells as obtained by the method of the disclosure also exhibit telomerase activity: They express TERT gene at a level which correlates with telomerase activity. This activity can be detected under similar conditions as murine ES cells, which is known to express significant telomerase activity as compared to somatic fibroblast prior to reprogramming.

The reprogrammed stem cells are further characterized in that they proliferate rapidly (in particular more than twice faster as their original somatic cells) and have a G0/G1 phase similar to embryonic stem cells (i.e. much shorter than somatic cells). However, they have certain features that clearly distinguish from embryonic stem cells: In particular, they do not express the OCT4 and NANOG pluripotent genes

Their proliferative capacity is still observed after 150 days without any modification in the growth proliferation if maintained under identical culturing conditions and without any remarkable change of their phenotype.

The reprogrammed stem cells of the present disclosure also do show any endogenous expression of any of the following markers: OCT4, KLF4 and NANOG. However, they show exogenous expression of CDX-2, c-MYC and ESRRB genes.

These stem cell compositions typically may comprise reprogrammed stem cells obtained from human, bats or equine somatic cells.

In a specific embodiment, the reprogrammed stem cells of the disclosure are characterized in that their susceptibility to virus infection, for example Nipah virus infection, is increased up to at least 50%, particularly at least 90%, as compared to corresponding somatic cells, as measured in vitro by an infection test. Such infection test may be carried out as described below in the experimental part (Examples). Other virus that can be tested include without limitation including Bunyaviridae, Mononegaviridae with the Filovirus (Ebola, Marburg, . . . ); Paramyxoviridae with the Coronavirus (SRAS, MERS, . . . ), Henipavirus (NiV, HNV) families, Togaviridae, Flaviviridae, Rhabdoviridae, etc. . . .

In other specific embodiments, the reprogrammed stem cells are bat stem cells and have similar gene expression profile as the stem cells as deposited on Mar. 14, 2018, at the NCBI under deposit number CNCM 1-5295, in at least one or more of the following gene markers: TERT, EMA1, NELL2, SIDT1, NWD2, SOX2, TBR1.

In other specific embodiments, the reprogrammed stem cells are bovine stem cells and have similar gene expression profile as the stem cells as deposited on Mar. 14, 2018, at the NCBI under deposit number CNCM 1-5296, in at least one or more of the following markers: TERT, CDL1, DAB1, GMPR, FOLR1, PIWIL1, TOPAZ.

In other specific embodiments, the reprogrammed stem cells are human stem cells and have similar gene expression profile as the stem cells as deposited on Mar. 14, 2018, at the NCBI under deposit number CNCM 1-5297, in at least one or more of the following gene markers: TERT, KLFS, GATA4.

Expression profile of each marker is determined by measuring the relative gene expression compared to a control gene such as RLP17 or TBP, as housekeeping genes. As used herein, “a similar expression profile” means that the relative expression profile of a marker in a stem cell of the disclosure is identical (or +/−15% equal) to the relative expression profile as determined in one of the deposited stem cells, when cultured under similar conditions.

In specific embodiments, the reprogrammed stem cells are the cells as deposited on March 14, 2018, at the CNCM under deposit number CNCM 1-5295, CNCM 1-5296 or CNCM I-5297,

### Uses of the Stem Cells

The stem cells obtained or obtainable from the methods of the disclosure may advantageously be cultured in vitro under differentiation conditions, to generate differentiated cells, such as muscle, cartilage, bone, dermal tissue, cardiac or vascular tissue, or other tissues of interest.

The skilled person may use known protocols for differentiating stem cells, such as the protocols conventionally used for differentiating induced pluripotent stem cells, ES cells or mesenchymal stem cells into the desired cell lineages.

One major field of application is cell therapy or regenerative medicine.

For example, primary cells, such as fibroblast cells obtained from a subject suffering from a genetic defect, may be cultured and genetically corrected according to methods known in the art, and subsequently reprogrammed into stem cells according to the methods of the present disclosure and differentiated into the suitable cell lineages for re-administration into the subject, for example the same subject as the cell donor (autologous treatment).

Similarly, regenerative medicine can be used to potentially cure any disease that results from malfunctioning, damaged or failing tissue by either regenerating the damaged tissues in vivo by direct in vivo implanting of a composition comprising the stem cells or their derivatives comprising appropriate progenitors or cell lineages.

In one aspect, the reprogrammed stem cells may be useful for autologous regenerative therapy of a patient in need of regenerative therapy due to specific disorders or treatments associated to such disorders, including without limitation, cancer disorders, inflammatory and autoimmune disorders, muscle and skeletal disorders, neurologic disorders, diabete and other metabolic disorders.

In another specific embodiment, the reprogrammed stem cell compositions are used for the treatment of joint or cartilage, muscle or bone damages.

In another specific embodiment, the reprogrammed stem cell compositions may also be used advantageously for the production of dermal tissues, for example, skin tissues, for use in regenerative medicine (cell-based therapy) or in research.

In another specific embodiment, the reprogrammed stem cell compositions may also be used advantageously for the production of, but not restricted to, dermal, muscle or skeletal cells from healthy or diseased patients for screening applications in the pharmaceutical industry. Such screening tests can be used to search for new drugs with clinical applications or for toxicology tests.

In another specific embodiment, the reprogrammed stem cell compositions may also be used for regenerating cardiac or vascular tissue.

In another specific embodiment, the reprogrammed stem cell compositions may also be used for regenerating brain tissue or neuronal tissue, for example in patient suffering from neurodegenerative disorders.

In another specific embodiment, the reprogrammed stem cell compositions may also be used for replicating and testing the virulence of pathogens, in particular, viral pathogens, or as a research tool for studying infection and propagation of viral pathogens.

The disclosure will be further illustrated by the following examples. However, these examples should not be interpreted in any way as limiting the scope of the present invention.

### DESCRIPTION OF THE FIGURES

FIG. 1: Map of pPB-CAG-rtTA3-IRES-PURO-TRE-SV40pA plasmid for the conditional expression of genes using the rtTA3 system. In the presence of doxycycline, rtTA3 releases TRE (Tet Response Element) to enable transcription following the latter.

FIG. 2: Growth curve of PTC primary cells (circle) and cells preprogrammed using the ECM combination in a ESM2 (triangle) or EpiStem medium (square).

FIG. 3: Susceptibility of Pteropus bat reprogrammed cells (BRCs) to henipaviruses. Infection of bat primary cell (BPC), bat reprogrammed cells (BRC) and Vero cells with VSV pseudotyped Nipah virus glycoproteins from malaysian strain NiVM or Bangladesh strain-NiVB, or Hendra virus glycoproteins (HeV) at moi 0.1. The susceptibility was quantified thanks to reporter gene (RFP) content in VSV genome by fow cytometry.

FIG. 4: Henipaviruses infections and titration.

Analysis of Nipah viruses infections kinetics by NiV-N mRNA production by RT-qPCR and virions production with supernatant titration. Bat primary cells (BPC—plain lines) and bat reprogrammed cells (BRC—dotted lines) were infected at a MOI of 0.1 with three strains of Nipah viruses: NiV Malaysia virus (NiV M, isolate UMMC1; GenBank AY029767), NiV Bangladesh virus (NiV B, isolate SPB200401066, GenBank (AY988601) and NiV Cambodia virus (NiV C, isolate NiV/KHM/CSUR381). The viruses were prepared on Vero-E9 cells. At the indicated times, the transcription viral kinetics were quantified by RT-qPCR in cells.

### EXAMPLES

### EXAMPLE 1: Reprogramming of Bat Somatic Cells

### Primary Cultures of Bat Cells

Biopsies of Pteropus giganteus and Pteropus vampyrus, recognised as being natural reservoirs of Nipah virus (NiV), have been made from several tissues and explants derived from the trachea, lung and alary membrane, and have been put in culture either in Fibroblast Medium (FM) or in a ES cells medium (ESM1).

The FM medium is composed of DMEM/F12 (Gibco, 11320-033) supplemented with 10% foetal bovine serum (FBS), 1% L glutamine (200 mM) (Gibco, 25030-024), 1000 U/mL of penicillin, 1000 U/ mL of streptomycin (Gibco, 15140-122).

The ESM1 medium is composed of DMEM/F12 (Gibco, 11320-033) supplemented with 10% of FBS, 1% L glutamine (200 mM) (Gibco, 25030-024), 1000 U/mL of penicillin, 1000 U/ mL streptomycin (Gibco, 15140-122), 1% of non-essential amino acids (100×) (Gibco, 11140-035), 1% of sodium pyruvate (100 mM) (Gibco, 11360-070), 0.1 mM of β-mercaptoethanol (Gibco, 31350-010), 1 ng/ml of IL6 (Peprotech, Ref. 200-06), 1 ng/ml of IL6 receptor (Peprotech, Ref. 200-06R), 1 ng/ml Mouse Stem Cell Factor (mSCF) (Peprotech, Ref: 300-07), 5 ng/ml of insulin-like growth factor-1 (IGF1) (Peprotech, 100- 11) and 1000 U/ml of Leukemia inhibitory factor (LIF).

After adhesion of explants in a previously gelatinised 6-well culture plate, cells begin to come out after 5 days of culture for (Primary Trachea cells) cells of the trachea explant, after 30 days of culture for PLC (Primary Lung cells) cells from the lung explant and after 15 days culture for PTGV (Pteropus giganteus Vienna Zoo) cells of the alary membrane explant. After 7 days, proliferating cells are dissociated and reseeded at a concentration of 5×10⁴ cells per cm². Overall, the morphology of the cells is typical of fibroblasts with the appearance of an elongated more or less flattened cell, but with morphological differences observed as a function of the seeding medium.

In order to establish their long-term proliferation potential, the cells are dissociated at confluence in the presence of trypsin according to a classical protocol for the care of fibroblasts and maintained until entry to senescence, phenomenon observed by slowing of their proliferation potential and a morphological change with the appearance of giant cells with a cytoplasm that leads to progressive stopping of proliferation and disappearance of the culture.

At each passage, 2×10⁵ dissociated cells are seeded in 1 well of a 6-well plate in an FM or ESM1 medium as indicated and held in an incubator at 37° C., at 7.5% of CO2. The medium is changed every 2-3 days. When the cells reach confluence, they are rinsed with PBS and dissociated with TrypLE™ Express Enzyme (1×) (Gibco, 12604-013), the action of which is then stopped by the addition of a complete culture medium. After centrifuging at 400 g, the dissociated cells are recovered in the complete medium, counted and reseeded as described. The growth curves are established from the counts.

PTGV and PLC cells become senescent after 4 to 10 generations obtained after about 50 to 120 days of culture, while the PTC cells continue to proliferate after 31 generations and more than 200 days of continuous culture.

The medium influences the morphology and also the proliferation of cells. The ESM1 medium enables a larger number of generations for the same number of PLC culture days.

### Constructions of Inducible Transposon Vectors

The P. giganteus genome is not available while the P. vampyrus genome is only partially annotated, particularly in comparison with the human genome that is much better known.

By comparing the encoding phases of human and bat pluripotent genes OCT4, SOX2, KLF4, c-MYC and NANOG and the two genes CDX2 and ESRRB used in the particular combinations in the application, it is found that the protein alignments evaluated using EXPASY (http://web.expasy.org/sim/) are very close at between 87 and 99% except for the NANOG gene with almost 70% alignment (see Tables 2 and 3). Most constructions were made using available human encoding phases.

| Gene | | P. Vampyrus | |
|---|---|---|---|
| | Gene ID | mRNA | Protein |
| OCT4 | 105308128 | XM_011383909.1 | XP_011382211.1 |
| SOX2 | 105307378 | XM_011382865.1 | XP_011381167.1 |
| KLF4 | 105295332 | XM_011364950.1 | XP_011363252.1 |
| C-MYC | 105292561 | XM_011360819.1 | XP_011359121.1 |
| NANOG | 105295921 | XM_011365777.1 | XP_011364079.1 |
| CDX2 | 105292134, | XM_011360237.1 | XP_011358539.1 |
| ESRRB | 105290083 | XM_011357045.1 | XP_011355347.1 |

| | | | | % align- |
|---|---|---|---|---|
| | | | | ment |
| | | | | (proteins) |
| | H. Sapiens | | | between P. |
| Gene | Gene ID | mRNA | Protein | [symbol] |
| OCT4 | 5460 | NM_002701.5 | NP_002692.2 | 90.9 |
| SOX2 | 6657 | NM_003106.3 | NP_003097.1 | 99.1 |
| KLF4 | 9314 | NM_001314052.1 | NP_001300981.1 | 87.3 |
| C-MYC | 4609 | NM_002467.4 | NP_002458.2 | 92.0 |
| NANOG | 79923 | NM_024865.3 | NP_079141.2 | 69.9 |
| CDX2 | 1045 | NM_001265.4 | NP_001256.3 | 91.0 |
| ESRRB | 2103 | NM_004452.3 | NP_004443.3 | 98.8 |

[symbol]

indicates data missing or illegible when filed

Tables 2 and 3: Comparison of nucleotide and protein sequences of pluripotent genes between Pteropus vampyrus bat and man.

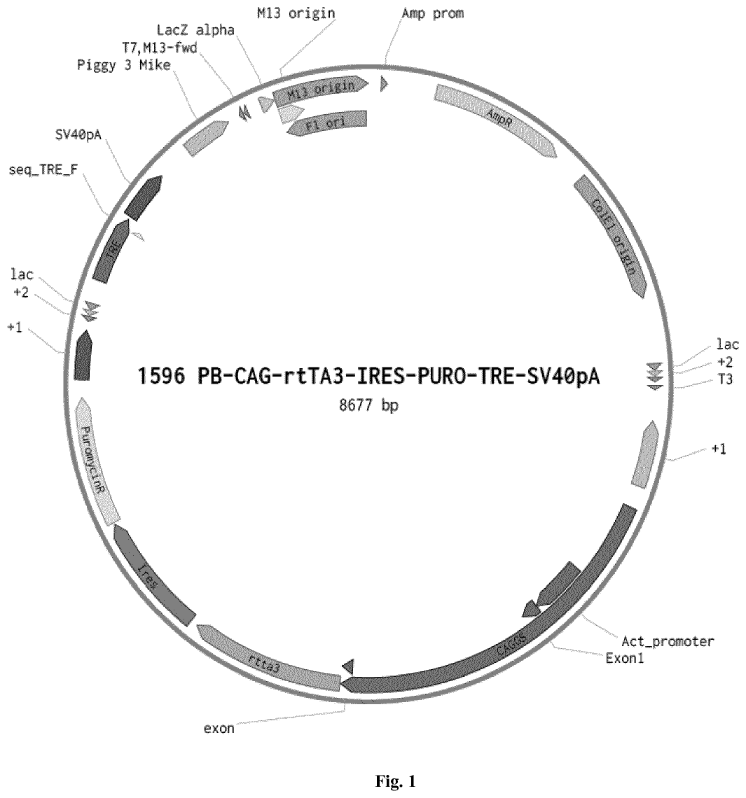

The skeleton of the vector used is a pPB transposon modified from the original vector to enable a conditional expression making use of the rtTA3 system as described (FIG. 1). Each cDNA is inserted using a NEBuilder® HiFi DNA Assembly Master Mix system (E2621, BioLabs) using the oligonucleotides listed in Table 4.

TABLE 4
List of oligos necessary for cloning of different human, mouse and bovine reprogramming
genes, in the pPB-CAG-rtTA3-IRES-PURO-TRE-SV40pA plasmid.

| Plasmid pPB | Primers | Sequence |
|---|---|---|
| PB CAG rtTA3 IP TRE hSox2 SV40pA | Sense | Ccactagtcgagttaattatgtacaacatgatggagacg (SEQ ID NO: 1) |
| | Anti-sense | Gatcagttatctagattaattcacatgtgtgagagggg (SEQ ID NO: 2) |
| PB CAG rtTA3 IP TRE hPou5f1 | Sense | Ccactagtcgagttaattatggcgggacacctggct (SEQ ID NO: 3) |
| SV40pA | Anti-sense | Gatcagttatctagattaattcagtttgaatgcatgggagag (SEQ ID NO: 4) |
| PB CAG rtTA3 IP TRE hK1f4 SV40pA | Sense | Ccactagtcgagttaattatgaggcagccacctggc (SEQ ID NO: 5) |
| | Anti-sense | Gatcagttatctagattaatttaaaaatgcctcttcatgtgtaagg (SEQ ID |
| | | NO: 6) |
| PB CAG rtTA3 IP TRE h-Myc SV40pA | Sense | Ccactagtcgagttaattatgcccctcaacgttagc (SEQ ID NO: 7) |
| | Anti-sense | Gatcagttatctagattaatttacgcacaagagttccg (SEQ ID NO: 8) |
| PB CAG rtTA3 IP TRE hNanog | Sense | Ccactagtcgagttaattatgagtgtggatccagcttg (SEQ ID NO: 9) |
| SV40pA | Anti-sense | Gatcagttatctagattaattcaggttgcatgttcatg (SEQ ID NO: 10) |
| PB CAG rtTA3 IP TRE hCDX2 SV40pA | Sense | Ccactagtcgagttaattatgtacgtgagctacctcctg (SEQ ID NO: 11) |
| | Anti-sense | gatcagttatctagattaatTCACTGGGTGACGGTGGG (SEQ ID |
| | | NO: 12) |
| PB CAG rtTA3 IP TRE mEsrrB | Sense | Ccactagtcgagttaattatgctgctgaaccgaatg (SEQ ID NO: 13) |
| SV40pA | Anti-sense | Gatcagttatctagattaattcacaccttggcctccag (SEQ ID NO: 14) |
| PB CAG rtTA3 IP TRE bc-Myc SV40pA | Sense | ttgagagcaaccctggacctATGCCCCTCAACGTCAGC (SEQ |
| | | ID NO: 15) |
| | Anti-sense | attatgatcagttatctagaTTAGGCGCAAGAGTTCCG (SEQ ID |
| | | NO: 16) |

### Attempt at Reprogramming Bat Somatic Cells by the OSKM Combination

Different reprogramming tests were carried out on bat cells at different early passages after cells are extracted from the explant (<pass 10).

A first test was carried out with the classical OSKM combination introduced by the Sendai virus in non-integrative form. 2×10⁵ PTGV cells seeded in a 6-well plate were infected with 5 MOIs for the two viruses containing KLF4-OCT4-SOX2 and c-MYC and one 3 MOI for the virus containing KLF4 according to the protocol described by the virus supplier—CytoTune2.0 (Invitrogen, A16517, A16518). Cells were passed 5 days after the infection and seeded in two 55 cm² boxes. The cells were put into to an ESM1 or EpiStem medium, 6 days after infection and the medium was changed every 2 days.

The “EpiStem” medium is an aseric medium composed of 50% of DMEM/F12 (Gibco, 11320-033) and 50% of Neurobasal medium (Gibco, 21103-049). It is supplemented by B-27 Supplement (50×) (Gibco, 17504-044), N-2 Supplement (5×) (Gibco, 17502048), 1% L-Glutamine (200 mM) (Gibco, 25030-024), 1000 U/mL penicillin, 1000 U/mL streptomycin (Gibco, 15140-122) and 1 mM β-mercapto-ethanol (Gibco, 31350-010). 5 ng/mL of β-Fibroblast Growth Factor (b-FGF) (Peprotech, 100-18B) and 10 ng/mL of human Activin A (Peprotech, 120-14E) are added to this medium.

Virus detection tests done by PCR as recommended by the supplier one week after the infection at the time of the first pass show that the cells were well infected. There were some morphological changes in the ESM1 medium that appeared in concentrated clusters, but they were transient and not maintained. The aseric EpiStem medium cannot be used to maintain cells in culture.

Infected cells cultivated in the ESM1 medium were kept for more than 5 months without any important change occurring, either morphologically or in proliferation of cells that have entered senescence. The presence of NANOG in cells at the time of infection did not modify the kinetics or the absence of any major morphological modification.

No ‘iPS-like’ cells are obtained from these primary cells under these conditions. iPS cells are easily obtained using this system in other species including in man.

In another embodiment, the cells were modified by electroporation with inducible transposons encoding for OCT4, SOX2, KLF4 and c-MYC in, the presence or absence of NANOG. PLC and PTC cells were dissociated, and centrifuged at 1200 rpm (300 g) at ambient temperature for 5 min. After suction of the float, the cell pellet was rinsed in PBS, centrifuged again and 1×10⁶ cells were directly recovered in 120 μL of resuspension buffer (Neon, Life Technologies, MPK5000). 2μg of transposase (⅓ of the total quantity plasmids) and 4 μg of vectors (⅔ of the total quantity of plasmids) in purified plasmid form were added to this mix, composed of different doxycycline-inducible Piggybac transposons for which the composition varies depending on the tested combination (Table 4). The plasmids mix was electroporated by the Neon system (Life Technologies, MPK5000), at 1500V, during 30 ms, in 1 pulse, in a 1000, cone, immersed in a tank containing electroporation buffer (Neon, Life Technologies, MPK5000). After electroporation, the cells were put into culture in a 6-well plate, in 3 mL of FM medium. The electroporated cells medium was replaced after 24 h and a selection was made by 5 μg/mL puromycin and by 200 μg/mL neomycin depending on the resistance genes carried by the plasmids present in the combination. The medium with selection was changed every two days for at least one week. At the end of the selection between 8 and 15 days, the cells were dissociated by 0.05% trypsin-EDTA(Life) and 2×10⁵ cells were seeded in a well in a 6-well plate, in 3 mL of ESM1 or ESM2 medium to which 2 ug/mL of doxycycline was added.

The “ESM2” medium is composed of DMEM/F12 (Gibco, 11320-033) supplemented by 10% of foetal bovine serum (FBS), 1% L glutamine (200 mM) (Gibco, 25030-024), 1000 U/mL of penicillin, 1000 U/ mL of streptomycin (Gibco, 15140-122), 1% of non-essential amino acids (100×) (Gibco, 11140-035), 1% of sodium pyruvate (100 mM) (Gicbo, 11360), 0.1 mM de f3-mercaptoethanol (Gibco, 31350-010), 5 ng/mL of b-FGF (Peprotech, 100-18B) and 10 ng/mL of human Activin A (Peprotech, 120-14E).

Only transient morphological changes occured in the “ESM1” and “ESM2” media. Cells were grouped in clusters as observed during the infection by the Sendai virus and sometimes become smaller. These morphological changes were lost after the first pass.

TABLE 5
Combinations of reprogramming genes tested in
bat cells by electroporation of transposons.

| Com- | Reprogramming | | % alignment with the | Cell |
|---|---|---|---|---|
| bination | gene | Species | P. vampyrus protein | cultures |
| OSKM | OCT4 | Human | 90.9 | PTGV |
| | SOX2 | Human | 99.1 | PLC |
| | KLF4 | Human | 87.3 | PTC |
| | c-MYC | Human | 92 | |
| OSKMN | OCT4 | Human | 90.9 | PTGV |
| | SOX2 | Human | 99.1 | PLC |
| | KLF4 | Human | 87.3 | PTC |
| | c-MYC | Human | 92 | |
| | NANOG | Human | 69.9 | |
| ECM | ESRRB | Mouse | 92.4 | PTC |
| | (NP_036064.3) | | | |
| | CDX2 | Human | 91 | |
| | c-MYC | Bovine | 95.0 | |
| | (NP_ | | | |
| | 001039539.1) | | | |

### ECM Reprogramming in Bat Somatic Cells

In previous experiments, the combination of CDX2 and c-MYC genes had been identified as providing a means of obtaining stem cells in different species such as ruminants (bovine, goat, sheep, etc.) and also in pigs.

Using the same protocol as that described above, PTC cells were electroporated with inducible transposons containing CDX2, c-MYC and ESRRB genes. At the end of the selection, the cells were dissociated by the TrypLE™ Express Enzyme (1×) (Gibco, 12604-013) and 2×10⁵ cells were seeded in a well in a 6-well plate, in 3 mL of ESM2 or EpiStem medium to which Zug/ml of doxycycline was added.

This new combination to which the ESRRB gene was added enabled the appearance of major morphological changes in PTCs. In the “EpiStem” medium, morphological changes appeared on average starting 20 days after the addition of doxycycline, while at least 30 days were necessary for the “ESM2” medium. Colonies of modified cells were sampled after 35 days of induction and seeded in gelatined wells in 24-well plates.

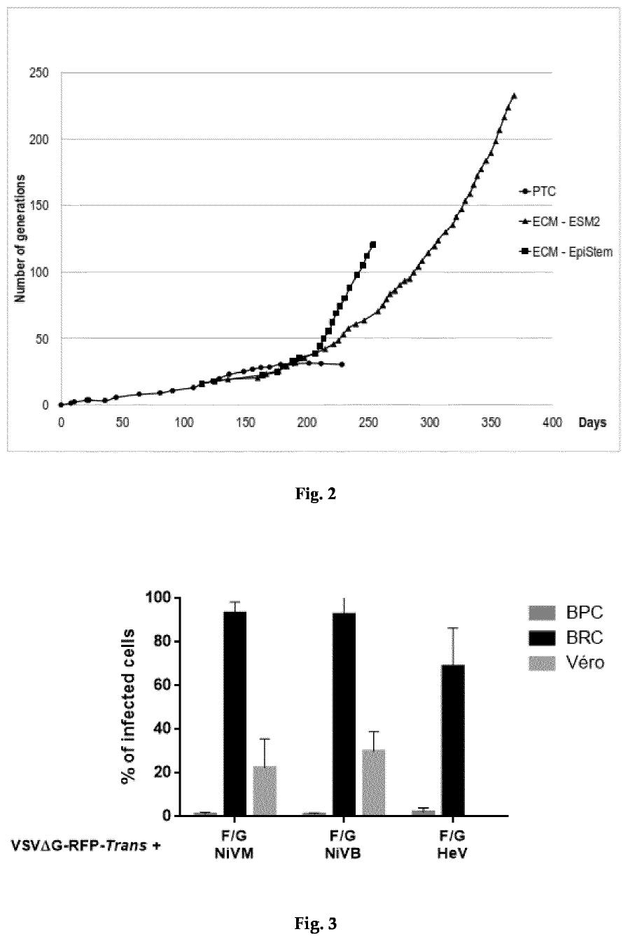

The proliferation of cells was very different from the proliferation of PTCs (FIG. 2). While a plateau for PTCs is reached at 31 generations and 200 days of continuous culture, reprogrammed cells have high growth without reaching a plateau after 30 to 45 generations and 223 days of continuous culture.

### Infection Test

Reprogrammed and primary cells are tested for replication of NiV. The susceptibility of cells is evaluated using a recombining NiV virus suitable for the expression of GFP. This virus was produced by reverse genetics. The GFP gene was introduced into a plasmid containing the NiV genome, between the N (nucleoprotein) and P (phosphoprotein) viral genes. This genomic plasmid is co-transfected with a mix of plasmid coding for viral proteins forming the replication complex (N, P and L) in CV-1 cells expressing polymerase T7. The recombining virus has characteristics similar to the parent virus (Yoneda et al., 2006). The virus is then produced on Vero-E6 cells.

NiV is a highly pathogenic virus for which there is as yet no validated vaccine or treatment, therefore its manipulation is restricted to biosecurity level 4 laboratories. The following infections are made in the Jean Merieux biosecurity level 4 laboratory in Lyon.

Porcine, bovine and bat primary cells are seeded in an FM medium with 2×10⁵ cells per well in a 12-well plate. Cells reprogrammed using OSKM combination (porcine) or the ECM combination (bovine and bat) are seeded in an ESM1, ESM2, EpiStem or KS medium with 2×10⁵ cells per well in a 12-well plate.

The “KS” medium is composed of DMEM/F12 (Gicbo, 11320-033) supplemented with 1000 U/mL of penicillin, 1000 U/ mL of streptomycin (Gibco, 15140-122), 1% of Insulin-Transferrin-Selenium (ITS) (100×) (Gibco, 41400-045), 10-6M of 3,3′,5-Triiodo-L-thyronine sodium salt (L-T3) (Sigma, T6367), 0.5 μg/ml of hydrocortisone (Sigma, H0888-1G), 0.3 nM of L-ascorbic acid (Sigma, A92902), 5 ng/ml of hBMP4 (Peprotech, 120-05ET), 5 ng/ml of hKGF (Peprotech, 100-19) and 5 ng/ml of mEGF (Peprotech, 315-09).

The infection is made 24 hours after seeding with an MOI of 3 in a 0% DMEM medium during lh at 37° C. and with 5% of CO2. After the hour of infection, the cells are washed with 0% DMEM and the corresponding media are then added to the different cell types. The cells are incubated at 37° C. under 5% CO2. 24 after infection, the cells are rinsed with PBS and dissociated with TrypLE™ Express Enzyme (1×), the action of which is then stopped by the addition of a complete culture medium. After centrifuging at 400 g, the dissociated cells are fixed with 4% PFA for 30 min. The fluorescence analysis is made using a Navios flow cytometer (BECKMAN COULTER, DS-14644A).

Somatic reprogramming of primary porcine, bovine and bat cells increases the susceptibility of cells to NiV by up to 90% compared with primary cells. The composition of the medium does not appear to modulate the infection. In pigs, the infection of infected primary cells is increased from 2% to 40 to 98% in the most susceptible reprogrammed cells. In bovine, cells reprogrammed with the ECM combination are 40% more susceptible than primary cells. Finally, in bats, reprogrammed ECM cells induced in an EpiStem medium can be infected by NiV from 40 to 80% while PTC primaries hardly have 1% of infection. Somatic reprogramming of porcine, bovine and bat cells can increase the susceptibility of cells to NiV.

### Example 2: ECM Reprogramming of Bovine Somatic Cells

### Primary Cultures of Bovine Cells

Biopsies of Bos taurus have been made from explants derived from D60 foetus, and have been put in culture either in Fibroblast Medium (FM).

The FM medium is composed of DMEM/F12 (Gibco, 11320-033) supplemented with 10% foetal bovine serum (FBS), 1% L-glutamine (200 mM) (Gibco, 25030-024), 1000 U/mL of penicillin, 1000 U/ mL of streptomycin (Gibco, 15140-122).

In order to establish their long-term proliferation potential, the cells are dissociated at confluence in the presence of trypsin according to a classical protocol for the care of fibroblasts and maintained until entry to senescence, phenomenon observed by slowing of their proliferation potential and a morphological change with the appearance of giant cells with a cytoplasm that leads to progressive stopping of proliferation and disappearance of the culture.

At each passage, 1×10⁶ dissociated cells are seeded in 1 dish of 100 mm in an FM and held in an incubator at 38.5° C., at 7.5% of CO₂. The medium is changed every 2-3 days. When the cells reach confluence, they are rinsed with PBS and dissociated with Trypsin-EDTA 1×, the action of which is then stopped by the addition of a complete culture medium.

After centrifuging at 400g, the dissociated cells are recovered in the complete medium, counted and reseeded as described. The growth curves are established from the counts.

### Construction of Inducible Transposon Vectors

| | B. taurus | | | |
|---|---|---|---|---|
| Gene | Gene ID | mRNA | | Protein |
| OCT4 | ENSBTAG00000021111 | ENSBTAT00000028122.5 | | F1N017 |
| SOX2 | ENSBTAG00000011598 | ENSBTAT00000015411.5 | | A2VDX8 |
| KLF4 | ENSBTAG00000020355 | ENSBTAT00000027125.5 | | A7YWE2 |
| C-MYC | ENSBTAG00000008409 | ENSBTAT00000011066.4 | | Q2HJ27 |
| NANOG | ENSBTAG00000020916 | ENSBTAT00000027863.3 | | Q4JM65 |
| CDX2 | ENSBTAG00000001819 | ENSBTAT00000047376.3 | | F1MJX0 |
| ESRRB | ENSBTAG00000012285 | ENSBTAT00000016290.5 | | F1N0K9 |
| | | | | % alignment |
| | | | | (proteins) |
| | | | | between |
| | H. Sapiens | | | B. taurus and H. |
| Gene | Gene ID | mRNA | Protein | Sapiens species |
| OCT4 | 5460 | NM_002701.5 | NP_002692.2 | ? |
| SOX2 | 6657 | NM_003106.3 | NP_003097.1 | 99 |
| KLF4 | 9314 | NM_001314052.1 | NP_001300981.1 | 94 |
| C-MYC | 4609 | NM_002467.4 | NP_002458.2 | 91 |
| NANOG | 79923 | NM_024865.3 | NP_079141.2 | 71 |
| CDX2 | 1045 | NM_001265.4 | NP_001256.3 | 95 |
| ESRRB | 2103 | NM_004452.3 | NP_004443.3 | 88 |

| Plasmide pPB | Primers | Séquence |
|---|---|---|
| PB CAG rtTA3 IP TRE | Sens | ttgagagcaaccctggacctATGTACAACATGATGG |
| tagBFP bSox2 SV40pA | | AGACGG (SEQ ID NO: 17) |
| | Anti-sens | attatgatcagttatctagaTCACATGTGCGAGAGGG |
| | | G (SEQ ID NO :18) |
| PB CAG rtTA3 IP TRE | Sens | ttgagagcaaccctggacctATGGCGGGACACCTCG |
| mKO bPou5f1 SV40pA | | CT (SEQ ID NO: 19) |
| | Anti-sens | attatgatcagttatctagaTCAGTTTGAATGCATAG |
| | | GAGAGCC (SEQ ID NO: 20) |
| PB CAG rtTA3 IP TRE | Sens | ttgagagcaaccctggacctATGGAGAAGTACCTGA |
| eGFP bK1f5 SV40pA | | CACC (SEQ ID NO: 21) |
| | Anti-sens | attatgatcagttatctagaTTAGTTCTGGTGCCTCTT |
| | | C (SEQ ID NO: 22) |
| PB CAG rtTA3 IP TRE- | Sens | ttgagagcaaccctggacctATGCCCCTCAACGTCA |
| miRFP-b-Myc SV40pA | | GC (SEQ ID NO: 23) |
| | Anti-sens | attatgatcagttatctagaTTAGGCGCAAGAGTTCC |
| | | G (SEQ ID NO: 24) |
| PB CAG rtTA3 IP TRE | Sens | ttgagagcaaccctggacctATGAGTGTGGGCCCAG |
| mKO bNanog SV40pA | | CTTG (SEQ ID NO: 25) |
| | Anti-sens | attatgatcagttatctagaTTACAAATCTTCAGGCT |
| | | GTATGTTGAG (SEQ ID NO: 26) |
| PB CAG rtTA3 INEO3 | Sens | Ccactagtcgagttaattatgtacgtgagctacctcctg (SEQ |
| TRE hCDX2 SV40pA | | ID NO: 27) |
| | Anti-sens | gatcagttatctagattaatTCACTGGGTGACGGTGG |
| | | G (SEQ ID NO: 28) |
| PB CAG rtTA3 INEO3 | Sens | Ccactagtcgagttaattatgctgctgaaccgaatg (SEQ ID |
| TRE mEsrrB SV40pA | | NO: 29) |
| | Anti-sens | Gatcagttatctagattaattcacaccttggcctccag (SEQ ID |
| | | NO: 30) |

### Tables 6 and 7

### ECM Reprogramming and Characterization of the ECM Bovine Stem Cells

In previous experiments, the combination of CDX2 and c-MYC genes had been identified as providing a means of obtaining stem cells in different species such as ruminants (bovine, goat, sheep, etc.) and also in pigs.

Using the same protocol as that described above, BEF cells were electroporated with inducible transposons containing CDX2, c-MYC and ESRRB genes. At the end of the selection, the cells were dissociated by the Trypsin (1×) and 2×10⁵ cells were seeded in a well in a 6-well plate, in 3 mL of ES medium or EpiStem medium to which lug/mL of doxycycline was added.

The “ES” medium is composed of DMEM supplemented by 10% of foetal bovine serum (FBS), 1% L-glutamine (200 mM) (Gibco, 25030-024), 1000 U/mL of penicillin, 1000 U/mL of streptomycin (Gibco, 15140-122), 1% of sodium pyruvate (100 mM) (Gicbo, 11360), 0.1 mM de β-mercaptoethanol (Gibco, 31350-010), 5 ng/mL of b-FGF (Peprotech, 100-18B).

This new combination to which the ESRRB gene was added enabled the appearance of major morphological changes in BEF. In the “EpiStem” medium, morphological changes appeared on average starting 5 days after the addition of doxycycline, while at least 7 days were necessary for the “ES” medium. Colonies of modified cells were sampled after 7 days of induction and seeded in gelatined wells in 24-well plates.

### Example 3: ECM Reprogramming of Human Somatic Cells

### Primary Cultures of Human Cells

Biopsies taken during chirurgical procedure with ethical consent have been put in culture either in Fibroblast Medium (FM).

The FM medium is composed of DMEM/F12 (Gibco, 11320-033) supplemented with 10% foetal bovine serum (FBS), 1% L-glutamine (200 mM) (Gibco, 25030-024), 1000 U/mL of penicillin, 1000 U/ mL of streptomycin (Gibco, 15140-122).

In order to establish their long-term proliferation potential, the cells are dissociated at confluence in the presence of trypsin according to a classical protocol for the care of fibroblasts and maintained until entry to senescence, phenomenon observed by slowing of their proliferation potential and a morphological change with the appearance of giant cells with a cytoplasm that leads to progressive stopping of proliferation and disappearance of the culture.

At each passage, 1×10⁶ dissociated cells are seeded in 1 dish of 100 mm in an FM and held in an incubator at 38.5° C., at 7.5% of CO₂. The medium is changed every 2-3 days. When the cells reach confluence, they are rinsed with PBS and dissociated with Trypsin-EDTA 1×, the action of which is then stopped by the addition of a complete culture medium. After centrifuging at 400g, the dissociated cells are recovered in the complete medium, counted and reseeded as described. The growth curves are established from the counts

### Construction of Inducible Transposon Vectors

TABLE 8

| Plasmide pPB | Primers | Séquence |
|---|---|---|
| PB CAG rtTA3 IP TRE | Sens | Ccactagtcgagttaattatgtacaacatgatggagacg (SEQ ID |
| hSox2 SV40pA | | NO: 31) |
| | Anti-sens | Gatcagttatctagattaattcacatgtgtgagagggg (SEQ ID |
| | | NO: 32) |
| PB CAG rtTA3 IP TRE | Sens | Ccactagtcgagttaattatggcgggacacctggct (SEQ ID |
| hPou5f1 SV40pA | | NO: 33) |
| | Anti-sens | Gatcagttatctagattaattcagtttgaatgcatgggagag (SEQ ID |
| | | NO: 34) |
| PB CAG rtTA3 IP TRE | Sens | Ccactagtcgagttaattatgaggcagccacctggc (SEQ ID |
| hK1f4 SV40pA | | NO: 35) |
| | Anti-sens | Gatcagttatctagattaatttaaaaatgcctcttcatgtgtaagg (SEQ |
| | | ID NO: 36) |
| PB CAG rtTA3 IP TRE | Sens | Ccactagtcgagttaattatgcccctcaacgttagc (SEQ ID |
| h-Myc SV40pA | | NO: 37) |
| | Anti-sens | Gatcagttatctagattaatttacgcacaagagttccg (SEQ ID |
| | | NO: 39) |
| PB CAG rtTA3 INEO3 | Sens | Ccactagtcgagttaattatgagtgtggatccagcttg (SEQ ID |
| TRE hNanog SV40pA | | NO: 40) |
| | Anti-sens | Gatcagttatctagattaattcaggttgcatgttcatg (SEQ ID |
| | | NO: 41) |
| PB CAG rtTA3 INEO3 | Sens | Ccactagtcgagttaattatgtacgtgagctacctcctg (SEQ ID |
| TRE hCDX2 SV40pA | | NO: 42) |
| | Anti-sens | gatcagttatctagattaatTCACTGGGTGACGGTGGG |
| | | (SEQ ID NO: 43) |
| PB CAG rtTA3 INEO3 | Sens | Ccactagtcgagttaattatgctgctgaaccgaatg (SEQ ID |
| TRE mEsrrB SV40pA | | NO: 44) |
| | Anti-sens | Gatcagttatctagattaattcacaccttggcctccag (SEQ ID |
| | | NO: 45) |

### ECM Reprogramming and Characterization of the ECM Human Stem Cells

In previous experiments, the combination of CDX2 and c-MYC genes had been identified as providing a means of obtaining stem cells in different species such as ruminants (bovine, goat, sheep, etc.) and also in pigs.

Using the same protocol as that described above, HEF cells were electroporated with inducible transposons containing CDX2, c-MYC and ESRRB genes. At the end of the selection, the cells were dissociated by the Trypsin (1×) and 2×10⁵ cells were seeded in a well in a 6-well plate, in 3 mL of ES medium or EpiStem or mTeSR medium to which 1 ug/mL of doxycycline was added.

The “ES” medium is composed of DMEM supplemented by 10% of foetal bovine serum (FBS), 1% L glutamine (200 mM) (Gibco, 25030-024), 1000 U/mL of penicillin, 1000 U/mL of streptomycin (Gibco, 15140-122), 1% of sodium pyruvate (100 mM) (Gicbo, 11360), 0.1 mM de β-mercaptoethanol (Gibco, 31350-010), 5 ng/mL of b-FGF (Peprotech, 100-18B).

This new combination to which the ESRRB gene was added enabled the appearance of major morphological changes in HEF. In the “EpiStem” medium, morphological changes appeared on average starting 5 days after the addition of doxycycline, while at least 7 days were necessary for the “ES” medium. Colonies of modified cells were sampled after 7 days of induction and seeded in gelatined wells in 24-well plates.

### Example 4: ECM Reprogramming of Horse Somatic Cells

### Primary Cultures of Horse Cells

Biopsies of Equus caballus, have been made from several tissues and explants derived from the placenta, ear and blood, and have been put in culture either in Fibroblast Medium (FM).

The FM medium is composed of DMEM/F12 (Gibco, 11320-033) supplemented with 10% foetal bovine serum (FBS), 1% L-glutamine (200 mM) (Gibco, 25030-024), 1000 U/mL of penicillin, 1000 U/ mL of streptomycin (Gibco, 15140-122).

After adhesion of explants in a previously gelatinised 6-well culture plate, cells begin to come out after 5 days of culture. After 14 days, proliferating cells are dissociated and reseeded at a concentration of 5×10⁴ cells per cm². Overall, the morphology of the cells is typical of fibroblasts with the appearance of an elongated more or less flattened cell, but with morphological differences observed as a function of tissue.

In order to establish their long-term proliferation potential, the cells are dissociated at confluence in the presence of trypsin according to a classical protocol for the care of fibroblasts and maintained until entry to senescence, phenomenon observed by slowing of their proliferation potential and a morphological change with the appearance of giant cells with a cytoplasm that leads to progressive stopping of proliferation and disappearance of the culture.

At each passage, 1×10⁶ dissociated cells are seeded in 1 dish in an FM medium as indicated and held in an incubator at 37° C., at 7.5% of CO₂. The medium is changed every 2-3 days. When the cells reach confluence, they are rinsed with PBS and dissociated with Trypsin-EDTA 1×, the action of which is then stopped by the addition of a complete culture medium. After centrifuging at 400 g, the dissociated cells are recovered in the complete medium, counted and reseeded as described. The growth curves are established from the counts.

### Construction of Inducible Transposon Vectors

| | Equus. caballus | | | H. sapiens | | |
|---|---|---|---|---|---|---|
| Gène | Gene ID | mRNA | Protein | Gene ID | mRNA | Protein |
| OCT4 | ENSECAG00000008967 | ENSECAT00000009247.1 | F6Y386 | 5460 | NM_002701.5 | NP_002692.2 |
| SOX2 | ENSECAG00000010653 | ENSECAT00000011080.1 | F6PUQ2 | 6657 | NM_003106.3 | NP_003097.1 |
| KLF4 | ENSECAG00000010613 | ENSECAT00000011058.1 | F6Q2H5 | 9314 | NM_001314052.1 | NP_001300981.1 |
| C-MYC | ENSECAG00000022059 | ENSECAT00000023507.1 | F6YAJ2 | 4609 | NM_002467.4 | NP_002458.2 |
| NANOG | ENSECAG00000012614 | ENSECAT00000013204.1 | F6PXN9 | 79923 | NM_024865.3 | NP_079141.2 |
| CDX2 | ENSECAG00000000707 | ENSECAT00000000573.1 | F7C6L9 | 1045 | NM_001265.4 | NP_001256.3 |
| ESRRB | ENSECAG00000011167 | ENSECAT00000011841.1 | F7AEP9 | 2103 | NM_004452.3 | NP_004443.3 |

TABLE 10

| Plasmide pPB | Primers | Séquence |
|---|---|---|
| PB CAG rtTA3 IP TRE | Sens | Ccactagtcgagttaattatgtacaacatgatggagacg (SEQ ID NO: |
| hSox2 SV40pA | | 46) |
| | Anti-sens | Gatcagttatctagattaattcacatgtgtgagagggg (SEQ ID NO: |
| | | 47) |
| PB CAG rtTA3 IP TRE | Sens | Ccactagtcgagttaattatggcgggacacctggct (SEQ ID NO: 48) |
| hPou5fl SV40pA | Anti-sens | Gatcagttatctagattaattcagtttgaatgcatgggagag (SEQ ID |
| | | NO: 49) |
| PB CAG rtTA3 IP TRE | Sens | Ccactagtcgagttaattatgaggcagccacctggc (SEQ ID NO: 50) |
| hK1f4 SV40pA | Anti-sens | Gatcagttatctagattaatttaaaaatgcctcttcatgtgtaagg (SEQ ID |
| | | NO: 51) |
| PB CAG rtTA3 IP TRE | Sens | Ccactagtcgagttaattatgcccctcaacgttagc (SEQ ID NO: 52) |
| h-Myc SV40pA | Anti-sens | Gatcagttatctagattaatttacgcacaagagttccg (SEQ ID NO: 53) |
| PB CAG rtTA3 INEO3 | Sens | Ccactagtcgagttaattatgagtgtggatccagcttg (SEQ ID NO: |
| | | 54) |
| TRE hNanog SV40pA | Anti-sens | Gatcagttatctagattaattcaggttgcatgttcatg (SEQ ID NO: 55) |
| PB CAG rtTA3 INEO3 | Sens | Ccactagtcgagttaattatgtacgtgagctacctcctg (SEQ ID NO: |
| TRE hCDX2 SV40pA | | 56) |
| | Anti-sens | gatcagttatctagattaatTCACTGGGTGACGGTGGG (SEQ |
| | | ID NO: 57) |
| PB CAG rtTA3 INEO3 | Sens | Ccactagtcgagttaattatgctgctgaaccgaatg (SEQ ID NO: 58) |
| TRE mEsrrB SV40pA | Anti-sens | Gatcagttatctagattaattcacaccttggcctccag (SEQ ID NO: 38) |

### ECM Reprogramming and Characterization of the ECM Horse Stem Cells

In previous experiments, the combination of CDX2 and c-MYC genes had been identified as providing a means of obtaining stem cells in different species such as ruminants (bovine, goat, sheep, etc.) and also in pigs.

Using the same protocol as that described above, HorseEF cells were electroporated with inducible transposons containing CDX2, c-MYC and ESRRB genes. At the end of the selection, the cells were dissociated by the Trypsin (1×) and 2×10⁵ cells were seeded in a well in a 6-well plate, in 3 mL of ES medium or EpiStem medium to which lug/ml of doxycycline was added.

The “ES” medium is composed of DMEM supplemented by 10% of foetal bovine serum (FBS), 1% L-glutamine (200 mM) (Gibco, 25030-024), 1000 U/mL of penicillin, 1000 U/mL of streptomycin (Gibco, 15140-122), 1% of sodium pyruvate (100 mM) (Gicbo, 11360), 0.1 mM de β-mercaptoethanol (Gibco, 31350-010), 5 ng/mL of b-FGF (Peprotech, 100-18B).

This new combination to which the ESRRB gene was added enabled the appearance of major morphological changes in HorseEF. In the “EpiStem” medium, morphological changes appeared on average starting 5 days after the addition of doxycycline, while at least 7 days were necessary for the “ES” medium. Colonies of modified cells were sampled after 7 days of induction and seeded in gelatined wells in 24-well plates.

### Example 5: Differentiation of ECM Stem Cells into Various Lineages

The reprogrammed cells are induced into differentiation into various lineages including the epithelial and endothelial ones.

For induction of the ECM reprogrammed cells into the epithelial lineage, a Keratinocyte medium (KS Medium) is used and composed of DMEM/F12 (Gicbo, 11320-033) supplemented with 1000 U/mL of penicillin, 1000 U/mL of streptomycin (Gibco, 15140-122), 1% of Insulin-Transferrin-Selenium (ITS) (100×) (Gibco, 41400-045), 10⁻⁶M of 3,3′,5-Triiodo-L-thyronine sodium salt (L-T3) (Sigma, T6367), 0.5 μg/mL of hydrocortisone (Sigma, H0888-1G), 0.3 nM of L-ascorbic acid (Sigma, A92902), 5 ng/mL of hBMP4 (Peprotech, 120-05ET), 5 ng/mL of hKGF (Peprotech, 100-19) and 5 ng/mL of mEGF (Peprotech, 315-09).

The cells are plated in regular growing medium after being passaged as previously described. The next day, the medium was changed to the KS medium in the absence of doxycycline or with a decrease amount of 0.05 μg /mL to 0.01 μg/mL of Doxycycline. Morphological changes are observed 2 to 5 days after the first addition of KS medium.

### Example 6: Susceptibility of Pteropus Bat Reprogrammed Cells (BRCs) to Henipaviruses

Infection of bat primary cell (BPC), bat reprogrammed cells (BRC) and Vero cells with VSV pseudotyped Nipah virus glycoproteins from malaysian strain NiVM or Bangladesh strain-NiVB , or Hendra virus glycoproteins (HeV) at moi 0.1. The susceptibility was quantified thanks to reporter gene (RFP) content in VSV genome by fow cytometry.

Henipavirus pseudotyped particles were made from the VSV-ΔG-RFP, a recombinant VSV derived from a full-length complementary DNA clone of the VSV Indiana serotype in which the G-protein envelope has been replaced with RFP (Reynard and Volchkov, 2015).

For pseudotyping, BSRT7 cells were transfected with plasmids encoding different henipaviruses glycoproteins (pCCAGS/HeV-F+pCCAGS/HeV-G, pCCAGS/NiVM-F+pCCAGS/NiVM-G, pCCAGS/NiVB-F+pCCAGS/NiVB-G, pCCAGS/HeV-F+pCCAGS/HeV-G) and 16 hrs post transfection, cells were infected with VSVAG-RFP-transG at an MOI of 0.3. One day later, supernatants were harvested, cleared from cell debris by low-speed centrifugation and viral particles were pelleted at 250,000 g for 2 hours. Virus stocks were titrated using a tissue culture inducing fluorescence 50 (TCIF50) titration assay. Pseudotyped viruses were named after the virus providing surface glycoproteins.

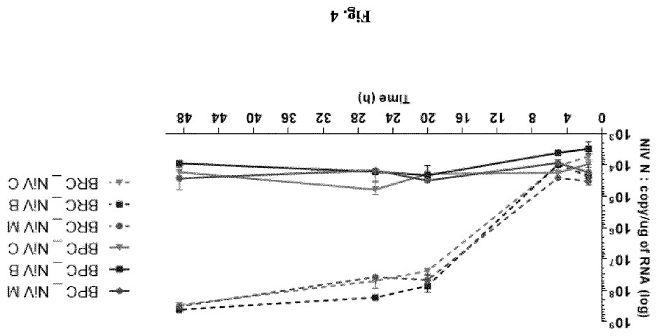

The results are shown in FIG. 3.

### Example 7: Henipaviruses Infections and Titration

Analysis of Nipah viruses infections kinetics by NiV-N mRNA production by RT-qPCR and virions production with supernatant titration. Bat primary cells (BPC) and bat reprogrammed cells (BRC) were infected with three strains of Nipah viruses: NiV Malaysia virus (isolate UMMC1; GenBank AY029767), NiV Bangladesh virus (isolate SPB200401066, GenBank (AY988601) and NiV Cambodia virus (isolate NiV/KHM/CSUR381). The viruses were prepared on Vero-E9 cells. Pteropus bat cells were infected at a MOI of 0.1. At the indicated times, the transcription viral kinetics were quantified by RT-qPCR in cells (see FIG. 4) while viral budding in supernants was analysed by viral titration.

### REFERENCES

Declercq J, Sheshadri P, Verfaillie C M, Kumar A. (2013); Zic3 enhances the generation of mouse induced pluripotent stem cells. Stem Cells Dev. 22: 2017-25.

Ezashi T, Telugu B P, Alexenko A P, Sachdev S, Sinha S, Roberts R M. (2009). Derivation of induced pluripotent stem cells from pig somatic cells. Proc Natl Acad Sci U S A. 106: 10993-8.

Feng B, Jiang J, Kraus P, Ng J H, Heng J C, Chan Y S, Yaw L P, Zhang W, Loh Y H, Han J, Vega V B, Cacheux-Rataboul V, Lim B, Lufkin T, Ng H H. (2009); Reprogramming of fibroblasts into induced pluripotent stem cells with orphan nuclear receptor Esrrb. Nat Cell Biol. 11: 197-203.

Han J, Yuan P, Yang H, Zhang J, Soh B S, Li P, Lim S L, Cao S, Tay J, Orlov Y L, Lufkin T, Ng H H, Tam W L, Lim B. (2010). Tbx3 improves the germ-line competency of induced pluripotent stem cells. Nature. 463 (7284):1096-100.

Heng J C, Feng B, Han J, Jiang J, Kraus P, Ng J H, Orlov Y L, Huss M, Yang L, Lufkin T, Lim B, Ng H H. (2010). The nuclear receptor Nr5a2 can replace Oct4 in the reprogramming of murine somatic cells to pluripotent cells. Cell Stem Cell. 6: 167-74.

Hochedlinger K, Plath K. (2009). Epigenetic reprogramming and induced pluripotency. Development. 136: 509-23. Review.

Honda A, Hatori M, Hirose M, Honda C, Izu H, Inoue K, Hirasawa R, Matoba S, Togayachi S, Miyoshi H, Ogura A. (2013). Naive-like conversion overcomes the limited differentiation capacity of induced pluripotent stem cells. J Biol Chem. 288: 26157-66

Iseki H, Nakachi Y, Hishida T, Yamashita-Sugahara Y, Hirasaki M, Ueda A, Tanimoto Y, Iijima S, Sugiyama F, Yagami K, Takahashi S, Okuda A, Okazaki Y. (2016). Combined Overexpression of JARID2, PRDM14, ESRRB, and SALL4A Dramatically Improves Efficiency and Kinetics of Reprogramming to Induced Pluripotent Stem Cells. Stem Cells. 34: 322-33.

Kaji K, Norrby K, Paca A, Mileikovsky M, Mohseni P, Woltjen K. (2009). Virus-free induction of pluripotency and subsequent excision of reprogramming factors. Nature. 458: 771-5.

Liu J, Balehosur D, Murray B, Kelly J M, Sumer H, Verma P J. (2012). Generation and characterization of reprogrammed sheep induced pluripotent stem cells. Theriogenology. 77: 338-46.

Nagy K, Sung H K, Zhang P, Laflamme S, Vincent P, Agha-Mohammadi S, Woltjen K, Monetti C, Michael I P, Smith L C, Nagy A. (2011). Induced pluripotent stem cell lines derived from equine fibroblasts. Stem Cell Rev.; 7 (3):693-702.

Mo X1, Li N1, Wu S2. Generation and characterization of bat-induced pluripotent stem cells. (2014). Theriogenology. 82: 283-93.

Osteil P, Tapponnier Y, Markossian S, Godet M, Schmaltz-Panneau B, Jouneau L, Cabau C, Joly T, Blachère T, Gócza E, Bernat A, Yerle M, Acloque H, Hidot S, Bosze Z, Duranthon V, Savatier P, Afanassieff M. (2013). Induced pluripotent stem cells derived from rabbits exhibit some characteristics of naïve pluripotency. Biol Open. 2: 613-28.

Reynard, O., and Volchkov, V. E. (2015). Characterization of a Novel Neutralizing monoclonal Antibody Against Ebola Virus GP. J. Infect. Dis. 212 Suppl 2, S372-378.

Shimada H, Nakada A, Hashimoto Y, Shigeno K, Shionoya Y, Nakamura T. (2010). Generation of canine induced pluripotent stem cells by retroviral transduction and chemical inhibitors. Mol Reprod Dev. 77: 2.

Sumer H, Liu J, Malaver-Ortega L F, Lim M L, Khodadadi K, Verma P J. (2011); NANOG is a key factor for induction of pluripotency in bovine adult fibroblasts. J Anim Sci. 89: 2708-16.

Takahashi K, Yamanaka S. (2006). Induction of pluripotent stem cells from mouse embryonic and adult fibroblast cultures by defined factors. Cell. 126: 663-76.

Takahashi K, Yamanaka S. (2016). A decade of transcription factor-mediated reprogramming to pluripotency. Nat Rev Mol Cell Biol. 17: 183-93.

Verma R, Liu J, Holland M K, Temple-Smith P, Williamson M, Verma P J. (2013). Nanog is an essential factor for induction of pluripotency in somatic cells from endangered felids. Biores Open Access. 2: 72-6.

Yoneda M1, Guillaume V, Ikeda F, Sakuma Y, Sato H, Wild T F, Kai C. Establishment of a Nipah virus rescue system. (2006). Proc Natl Acad Sci USA. 103: 16508-13.

Yu J, Vodyanik M A, Smuga-Otto K, Antosiewicz-Bourget J, Franc J L, Tian S, Nie J, Jonsdottir G A, Ruotti V, Stewart R, Slukvin II, Thomson J A. (2007); Induced pluripotent stem cell lines derived from human somatic cells. Science. 318: 1917-20.

1. An in vitro method for preparing stem cells reprogrammed from mammalian somatic cells, said method comprising culturing mammalian somatic cells and expressing at least the following combination of reprogramming factors:
i. a reprogramming factor encoded by ESRRB gene,
ii. a reprogramming factor encoded by CDX-2 gene, and,
iii. a reprogramming factor encoded by c-MYC gene,
under appropriate conditions for reprogramming said mammalian somatic cells into stem cells.

2. The method of claim 1, which does not include a step of exogenous expression of at least one of the following reprogramming factors: Oct4, Klf4, Sox2, Lin28 and Nanog.

3. The method of claim 1, wherein said mammalian somatic cells are selected from the group consisting of bovine somatic cells, equine somatic cells, human somatic cells and bat somatic cells.

4. The method of claim 1, wherein said mammalian somatic cells are selected from primary cells of blood, bone marrow, adipose tissue, skin, hair, skin appendages, internal organs mesenchymal tissues and parenchymal tissues containing primary fibroblasts, muscle, bone, cartilage or skeletal tissues.

5. The method of claim 1, wherein said appropriate conditions for reprogramming include the exogenous expression of the reprogramming factors for less than 50 days of culture.

6. The method according to claim 1, wherein said method comprises
a) introducing one or more expression vectors comprising nucleic acid sequences encoding said combination of reprogramming factors into said somatic cells; or,
b) directly delivering an effective amount of each reprogramming factor of the combination or precursor RNA encoding each reprogramming factor into said somatic cells.

7. The method of claim 6, wherein the step of introducing is performed by transfecting said somatic cells with at least one episomal, viral or transposon vector, or with a combination of episomal, viral and/or transposon vectors, comprising nucleic acid sequences encoding the reprogramming factors ESRRB, CDX-2 and c-MYC.

8. The method of claim 7, wherein the at least one transposon vector is an inducible transposon vector.

9. Reprogrammed Stem cells obtained according to the method of claim 1.

10. Reprogrammed Stem cells according to claim 9, wherein said stem cells are either
(i) bovine stem cells that express the telomerase gene TERT and at least one or more of the following genes: CDL1, DAB1, GMPR, FOLR1, PIWIL1, TOPAZ;
(ii) bat stem cells that express the telomerase gene TERT, and EMA1, and one or more of the following genes: NELL2, SIDT1, NWD2, SOX2, TBR1;
(iii) human stem cells that express the telomerase gene TERT and one or more of the following genes: KLF5, GATA4.

11. Reprogrammed Stem cells according to claim 9, characterized in that their susceptibility to virus infection is increased up to at least 50%, compared to parental somatic cells from which they have been obtained, as measured in vitro by an infection test.

12. Reprogrammed Stem cells according to, claim 9, as deposited at CNCM on Mar. 14, 2018, under deposit number CNCM I-5295, 1-5296 or 1-5297.

13. A kit for reprogramming mammalian somatic cells, said kit comprising:
one or more expression vectors for the exogenous expression of ESRRB, CDX-2 and c-MYC genes in mammalian cells, and
optionally, buffers and culturing media.

14. The kit of claim 13, wherein said expression vectors are selected from viral vectors, episomal vectors and transposon vectors.

15. (canceled)

16. The method of claim 4, wherein the internal organs include the heart, gut, lung, trachea, kidney or liver.

17. The method of claim 5, wherein the appropriate conditions for reprogramming include the exogenous expression of the reprogramming factors for from 5 to 10 days for the bovine cells somatic cells, from 10 to 20 days for the human and horse somatic cells and from 30 to 50 days for the bat somatic cells.

18. Reprogrammed Stem cells according to claim 11, characterized in that their susceptibility to virus infection is increased up to at least 90%.

19. A method of conducting cell or regenerative therapy in order to treat a genetic defect in a subject in need thereof comprising, obtaining primary cells from the subject;
obtaining genetically corrected cells by genetically correcting the genetic defect in the primary cells;
obtaining reprogrammed stem cells by reprogramming the genetically corrected cells into stem cells by the method of claim 1;
obtaining a differentiated cell-lineage by differentiating the reprogrammed stem cells; and
administering to the subject cells of the differentiated cell-lineage in an amount sufficient to treat the genetic defect.

20. The method of claim 19, wherein the primary cells are fibroblasts.


SEQUENCE LISTING (58 sequences)

SEQ ID NO: 1 - DNA, 39 bp, Homo sapiens
ccactagtcgagttaattatgtacaacatgatggagacg

SEQ ID NO: 2 - DNA, 38 bp, Homo sapiens
gatcagttatctagattaattcacatgtgtgagagggg

SEQ ID NO: 3 - DNA, 36 bp, Homo sapiens
ccactagtcgagttaattatggcgggacacctggct

SEQ ID NO: 4 - DNA, 42 bp, Homo sapiens
gatcagttatctagattaattcagtttgaatgcatgggagag

SEQ ID NO: 5 - DNA, 36 bp, Homo sapiens
ccactagtcgagttaattatgaggcagccacctggc

SEQ ID NO: 6 - DNA, 46 bp, Homo sapiens
gatcagttatctagattaatttaaaaatgcctcttcatgtgtaagg

SEQ ID NO: 7 - DNA, 36 bp, Homo sapiens
ccactagtcgagttaattatgcccctcaacgttagc

SEQ ID NO: 8 - DNA, 38 bp, Homo sapiens
gatcagttatctagattaatttacgcacaagagttccg

SEQ ID NO: 9 - DNA, 38 bp, Homo sapiens
ccactagtcgagttaattatgagtgtggatccagcttg

SEQ ID NO: 10 - DNA, 38 bp, Homo sapiens
gatcagttatctagattaattcaggttgcatgttcatg

SEQ ID NO: 11 - DNA, 39 bp, Homo sapiens
ccactagtcgagttaattatgtacgtgagctacctcctg

SEQ ID NO: 12 - DNA, 38 bp, Homo sapiens
gatcagttatctagattaattcactgggtgacggtggg

SEQ ID NO: 13 - DNA, 36 bp, Mus musculus
ccactagtcgagttaattatgctgctgaaccgaatg

SEQ ID NO: 14 - DNA, 38 bp, Mus musculus
gatcagttatctagattaattcacaccttggcctccag

SEQ ID NO: 15 - DNA, 38 bp, Bos taurus
ttgagagcaaccctggacctatgcccctcaacgtcagc

SEQ ID NO: 16 - DNA, 38 bp, Bos taurus
attatgatcagttatctagattaggcgcaagagttccg

SEQ ID NO: 17 - DNA, 42 bp, Bos taurus
ttgagagcaaccctggacctatgtacaacatgatggagacgg

SEQ ID NO: 18 - DNA, 38 bp, Bos taurus
attatgatcagttatctagatcacatgtgcgagagggg

SEQ ID NO: 19 - DNA, 38 bp, Bos taurus
ttgagagcaaccctggacctatggcgggacacctcgct

SEQ ID NO: 20 - DNA, 44 bp, Bos taurus
attatgatcagttatctagatcagtttgaatgcataggagagcc

SEQ ID NO: 21 - DNA, 40 bp, Bos taurus
ttgagagcaaccctggacctatggagaagtacctgacacc

SEQ ID NO: 22 - DNA, 39 bp, Bos taurus
attatgatcagttatctagattagttctggtgcctcttc

SEQ ID NO: 23 - DNA, 38 bp, Bos taurus; identical to SEQ ID NO: 15

SEQ ID NO: 24 - DNA, 38 bp, Bos taurus; identical to SEQ ID NO: 16

SEQ ID NO: 25 - DNA, 40 bp, Bos taurus
ttgagagcaaccctggacctatgagtgtgggcccagcttg

SEQ ID NO: 26 - DNA, 47 bp, Bos taurus
attatgatcagttatctagattacaaatcttcaggctgtatgttgag

SEQ ID NO: 27 - DNA, 39 bp, Bos taurus; identical to SEQ ID NO: 11

SEQ ID NO: 28 - DNA, 38 bp, Homo sapiens; identical to SEQ ID NO: 12

SEQ ID NO: 29 - DNA, 36 bp, Mus musculus; identical to SEQ ID NO: 13

SEQ ID NO: 30 - DNA, 38 bp, Mus musculus; identical to SEQ ID NO: 14

SEQ ID NO: 31 - DNA, 39 bp, Homo sapiens; identical to SEQ ID NO: 1

SEQ ID NO: 32 - DNA, 38 bp, Homo sapiens; identical to SEQ ID NO: 2

SEQ ID NO: 33 - DNA, 36 bp, Homo sapiens; identical to SEQ ID NO: 3

SEQ ID NO: 34 - DNA, 42 bp, Homo sapiens; identical to SEQ ID NO: 4

SEQ ID NO: 35 - DNA, 36 bp, Homo sapiens; identical to SEQ ID NO: 5

SEQ ID NO: 36 - DNA, 46 bp, Homo sapiens; identical to SEQ ID NO: 6

SEQ ID NO: 37 - DNA, 36 bp, Homo sapiens; identical to SEQ ID NO: 7

SEQ ID NO: 38 - DNA, 38 bp, Mus musculus; identical to SEQ ID NO: 14

SEQ ID NO: 39 - DNA, 38 bp, Homo sapiens; identical to SEQ ID NO: 8

SEQ ID NO: 40 - DNA, 38 bp, Homo sapiens; identical to SEQ ID NO: 9

SEQ ID NO: 41 - DNA, 38 bp, Homo sapiens; identical to SEQ ID NO: 10

SEQ ID NO: 42 - DNA, 39 bp, Homo sapiens; identical to SEQ ID NO: 11

SEQ ID NO: 43 - DNA, 38 bp, Homo sapiens; identical to SEQ ID NO: 12

SEQ ID NO: 44 - DNA, 36 bp, Mus musculus; identical to SEQ ID NO: 13

SEQ ID NO: 45 - DNA, 38 bp, Mus musculus; identical to SEQ ID NO: 14

SEQ ID NO: 46 - DNA, 39 bp, Homo sapiens; identical to SEQ ID NO: 1

SEQ ID NO: 47 - DNA, 38 bp, Homo sapiens; identical to SEQ ID NO: 2

SEQ ID NO: 48 - DNA, 36 bp, Homo sapiens; identical to SEQ ID NO: 3

SEQ ID NO: 49 - DNA, 42 bp, Homo sapiens; identical to SEQ ID NO: 4

SEQ ID NO: 50 - DNA, 36 bp, Homo sapiens; identical to SEQ ID NO: 5

SEQ ID NO: 51 - DNA, 46 bp, Homo sapiens; identical to SEQ ID NO: 6

SEQ ID NO: 52 - DNA, 36 bp, Homo sapiens; identical to SEQ ID NO: 7

SEQ ID NO: 53 - DNA, 38 bp, Homo sapiens; identical to SEQ ID NO: 8

SEQ ID NO: 54 - DNA, 38 bp, Homo sapiens; identical to SEQ ID NO: 9

SEQ ID NO: 55 - DNA, 38 bp, Homo sapiens; identical to SEQ ID NO: 10

SEQ ID NO: 56 - DNA, 39 bp, Homo sapiens; identical to SEQ ID NO: 11

SEQ ID NO: 57 - DNA, 38 bp, Homo sapiens; identical to SEQ ID NO: 12

SEQ ID NO: 58 - DNA, 36 bp, Mus musculus; identical to SEQ ID NO: 13